(19) United States
(12) Patent Application Publication
(10) Pub. No.: US 2020/0360072 A1
(43) Pub. Date: Nov. 19, 2020

(54) MICRONEEDLE TREATMENT SYSTEM

(71) Applicant: Patchmi, Inc., Manhattan Beach, CA (US)

(72) Inventors: Anna A. KUANG, Manhattan Beach, CA (US); Alissa M. FITZGERALD, San Francisco, CA (US)

(21) Appl. No.: 16/641,991

(22) PCT Filed: Aug. 28, 2018
(86) PCT No.: PCT/US2018/048394

§ 371 Date: Feb. 25, 2020

Related U.S. Application Data
(60) Provisional application No. 62/667,287, filed on May 4, 2018.
(60) Provisional application No. 62/551,655, filed on Aug. 29, 2017.

(51) Int. Cl. A61B 18/08; A61M 37/00; A61B 18/10

(52) U.S. Cl. CPC A61B 18/082; A61M 37/0015; A61B 18/10; A61B 2018/00464; A61M 2210/0612; A61M 2210/0606; A61M 2037/0061

(57) ABSTRACT

Described herein is a microneedle treatment system to reduce fat deposits directly under or in close proximity to skin, and to deliver energy or non-energy treatments to thicken and tighten dermis to treat skin laxity, wrinkles, improve skin scars, and other skin problems. The system can include a disposable patch with a microneedle array, and an overlying mask. The patch can be directly connected to a power source or an overlying mask can be configured to be placed directly over the disposable patch. The overlying mask can include a drive circuitry configured to deliver energy into the microneedle array, a sensor configured for localized sensing, and a telemetry uplink to smart phone, a computer or a computer network. Also described is a method to reduce fat deposits in close proximity to skin, and to deliver energy or non-energy treatments to thicken and tighten dermis using a microneedle array.

### CROSS REFERENCES TO RELATED APPLICATIONS

This application claims the benefit of U.S. Provisional Application No. 62/551,655, filed Aug. 29, 2017; and U.S. Provisional Application No. 62/667,287, filed May 4, 2018; the disclosures of each of which are incorporated herein by reference in their entities.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure relates generally to microneedle treatment systems and methods. In particular, the disclosure relates to microneedle treatment systems and related methods to deliver energy to reduce fat deposits directly under or in close proximity to the skin, and to deliver energy or non-energy treatments to thicken and tighten dermis to treat skin laxity, wrinkles, improve skin scars, and other skin problems.

### BACKGROUND

Aging is a natural process that is characterized by the development of areas of bulging fat and sagging skin. Anti-aging and beauty remedies are a billion-dollar industry which ranges from over-the-counter remedies to in-office minimally invasive and full surgical procedures. Skincare is one of the biggest segments in the beauty industry. The global skincare sales are estimated to be more than $130 billion by 2019. The global cosmetic surgery and services market is estimated to be more than $27 billion by 2019.

Currently, anti-aging treatments are often equated with surgical procedures. However, removing or reducing fat and tightening skin on the body usually requires cutting skin and using resection or cauterization to excise, melt, burn, cure, dissolve the fat, which are associated with pain, long recovery time, risk of anesthesia, risk with surgery, scars and increased cost. In general, surgery procedures, which are the current gold standard for anti-aging treatment, are invasive, costly and involve surgical and anesthetic risks. In addition, surgery procedures usually require three to six weeks recovery time.

There is a need for minimally invasive treatments such as microneedle systems that are small, pain free, cost-effective and available for home use.

### SUMMARY OF THE DISCLOSURE

Described herein is a microneedle treatment system for delivering energy to reduce fat deposits directly under or in close proximity to skin, and for delivering energy or non-energy treatments to thicken and tighten dermis to treat skin laxity, wrinkles, improve skin scars, and other skin problems. The microneedle treatment system can include a disposable patch and an overlying reusable mask. The disposable patch can include a microneedle array with a plurality of microneedles. The overlying mask can be configured to be placed directly over the disposable patch. The overlying mask can include a drive circuitry configured to deliver energy into the microneedle array, a sensor configured for localized sensing, and a telemetry uplink to a smartphone, a computer or a computer network. The overlying mask can also be battery-powered or powered directly by an electric control unit via an electrical wall plug cable.

In some embodiments each of the plurality of microneedles has a diameter between 100 μm to 200 μm. In some embodiments each of the plurality of microneedles has a length between 100 μm to 3500 μm. In some embodiments the disposable patch further comprises a micro-coil. In some embodiments the mask comprises a soft, flexible material. In some embodiments the mask and or patch further comprises a coil antenna to transfer power to the disposable patch by inductive power transfer. In some embodiments the mask further comprises a second antenna to send data from the mask to the internet, a nearby smartphone, or a nearby computer. In some embodiments the microneedle array can include multiple sub-arrays disposed onto multiple rigid substrates that form a semi-flexible substrate. In some embodiments the disposable patch can be customized.

Described herein is a method to reduce fat deposits in close proximity to skin, and to deliver energy or non-energy treatments to thicken and tighten dermis to treat skin laxity, wrinkles, improve skin scars, and other skin problems by using a microneedle treatment system. The method can include applying a disposable patch comprising a microneedle array to a targeted treatment area, placing an overlying mask directly over the disposable patch, delivering energy into the microneedle array, monitoring a heating function of the disposable patch, and transmitting data from the mask by using a telemetry uplink.

In some embodiments the method further includes receiving inductive power with a micro-coil on the disposable patch. In some embodiments the method further includes applying the energy through tips of the microneedles to a targeted area to reduce a targeted skin or fat layer. In some embodiments the method includes delivering treatment to targeted skin or fat layers without energy. In some embodiments the method includes use of insulated, coated or non-insulated microneedles. In some embodiments the method further includes delivering power inductively to the disposable patch from a coil antenna in the mask. In some embodiments the method further includes controlling the mask and the disposable patch by software application. In some embodiments the method further includes sending data from the mask to the internet, a nearby smartphone, or a nearby computer by a second antenna in the mask. In some embodiments the method further includes conforming the microneedle array to a large area of a body to be treated by using a microneedle array disposed on a series of rigid substrates that are tiled to form a semi-flexible substrate.

In some embodiments the step of applying the disposable patch includes applying the disposable patch under the eyes of a user in areas of fat deposits and skin laxity. In some embodiments the step of applying the disposable patch includes applying the disposable patch to jowls of a patient in areas of fat deposits and skin laxity. In some embodiments the step of applying the disposable patch includes applying the disposable patch to a nasolabial fold region of a patient in areas of fat deposits and skin laxity.

In some embodiments, there is a microneedle treatment system, comprising: a microneedle array attached to a patch, the microneedle array comprising a plurality of fixed-length microneedles, the microneedles comprising an insulated shaft and an uninsulated tip; and a power supply configured to heat the plurality of microneedles using less than about 2.5 W of power. In some embodiments, the power supply is configured to heat the plurality of microneedles using about 100 mW to about 1000 mW of power, about 100 mW to about 500 mW of power, or about 500 mW to about 1000 mW of power.

In some embodiments, there is a microneedle treatment system, comprising: a microneedle array attached to a patch, the microneedle array comprising a plurality of fixed-length microneedles, the microneedles comprising an insulated shaft and an uninsulated tip; and a power supply configured to heat the plurality of microneedles using about 50 mW of power or less per microneedle. In some embodiments, the power supply is configured to heat the plurality of microneedles using about 1 mW to about 50 mW of power per microneedle.

Also described herein is a microneedle treatment system, comprising: a patch comprising a dome-shape body comprising a top and a base, and a microneedle array comprising a plurality of microneedles housed within a cavity within the dome-shaped body and attached to an inner surface of the dome-shaped body, wherein the form of the body can be changed into a substantially flat configuration that results in at least a portion of the microneedles to be repositioned from within the cavity to below the base; and a power supply configured to heat the plurality of microneedles. In some embodiments, the microneedles are fixed-length microneedles. In some embodiments, the microneedles comprising an insulated shaft and an uninsulated tip. In some embodiments, the power supply is configured to heat the plurality of microneedles using less than about 2.5 W of power. In some embodiments, the power supply is configured to heat the plurality of microneedles using about 100 mW to about 1000 mW of power (such as about 100 mW to about 500 mW of power, or about 500 mW to about 100 mW of power). In some embodiments, the power supply is configured to heat the plurality of microneedles using about 50 mW of power or less per microneedle. In some embodiments, the power supply is configured to heat the plurality of microneedles using about 1 mW to about 50 mW of power per microneedle. In some embodiments, the base comprises a lip. In some embodiments, the base comprises an adhesive.

In some embodiments, the microneedles are about 2 mm to about 8 mm in length. In some embodiments, the microneedles are about 3 to about 4 mm in length. In some embodiments, the uninsulated tip is about 0.5 mm to about 1.0 mm in length. In some embodiments, the shaft of the microneedles is about 50 μm to about 500 μm in diameter.

In some embodiments, the plurality of microneedles comprises about 3 microneedles to about 100 microneedles.

In some embodiments, the power supply is configured to heat the tips of the microneedles from about 33° C. to about 60° C. In some embodiments, the plurality of microneedles is heated using a direct current energy. In some embodiment, the plurality of microneedles is heated using a radiofrequency energy.

In some embodiments, the system is a hands-free system. In some embodiments, the patch comprises an adhesive. In some embodiments, the patch is crescent-shaped, semi-circular, triangular, square, or rectangular. In some embodiments, the power supply comprises a battery. In some embodiments, the power supply is directly connected to the microneedle array through a wire. In some embodiments, the power supply is wirelessly connected to the microneedle array. In some embodiments, the patch comprises a first antenna electrically connected to the microneedle array, wherein the power supply comprises a second antenna, and wherein the power supply powers the microneedle array through inductive power transfer.

In some embodiments of the microneedle treatment system, the system comprises a mask comprising the power supply, wherein the mask is configured to be placed over the patch. In some embodiments, the mask is configured to be placed over, around, or below an eye of a human subject, and over the patch. In some embodiments, the patch or the mask comprises a temperature configured to suspend heating of the microneedles if the temperature goes above a predetermined threshold.

In some embodiments, the microneedle treatment system comprises a telemetry uplink antenna configured to communicate with a computer system or a network. In some embodiments, the system is operated using the computer system.

In some embodiments, there is a method of tightening skin or reducing a subcutaneous fat deposit of a subject, comprising: inserting the plurality of microneedles of any of the systems described above into the subject, wherein the tips of the microneedles are positioned within or on the surface of the subcutaneous fat deposit; and heating the tips of the microneedles, thereby melting fat within the facial fat area. In some embodiments, the subcutaneous fat deposit is a subcutaneous facial fat deposit. In some embodiments, the subcutaneous fat deposit is a postseptal fat deposit, a preseptal fat deposit, or a jowl fat deposit.

In some embodiments, there is a method of tightening skin or reducing a subcutaneous fat deposit in a subject, comprising: inserting a plurality of microneedles into a subject, wherein the tips of the microneedles are positioned within or on the surface of the subcutaneous fat deposit; heating the tips of the microneedles using less than about 2.5 W of power, thereby melting fat within the subcutaneous fat deposit. In some embodiments, heating the tips of the microneedles comprises applying about 100 mW to about 1000 mW of power (such as about 100 mW to about 500 mW of power, or about 500 mW to about 100 mW of power) to the microneedles.

In some embodiments, there is a method of reducing a subcutaneous fat deposit in a subject, comprising: inserting a plurality of microneedles into the subject, wherein the tips of the microneedles are positioned within or on the surface of the subcutaneous fat deposit; and heating the tips of the microneedles using about 50 mW of power or less per microneedle, thereby melting fat within the subcutaneous fat deposit. In some embodiments, heating the tips of the microneedles comprises applying about 1 mW to about 50 mW of power per microneedle.

In some embodiments, there is a method of reducing a subcutaneous fat deposit in a subject, comprising: positioning a dome-shaped patch comprising a plurality of microneedles on a target skin area above the subcutaneous fat deposit; reconfiguring the dome-shaped patch into a substantially flat configuration, thereby inserting the tips of the microneedles into the into the subcutaneous fat deposit; and heating the tips of the microneedles, thereby melting fat within the subcutaneous fat deposit. In some embodiments, reconfiguring the dome-shaped patch comprises applying pressure to the top of the dome-shaped patch. In some embodiments, the target skin area is stretched upon reconfiguring the dome-shaped patch into the substantially flat configuration.

In some embodiments, there is a method of tightening skin or reducing a facial fat deposit in a subject, comprising: inserting a plurality of microneedles into a subject, wherein the tips of the microneedles are positioned within or on the surface of the facial fat deposit; and heating the tips of the microneedles, thereby melting fat within the subcutaneous fat deposit. In some embodiments, the facial fat deposit is a periorbital postseptal fat deposit, a preseptal fat deposit, or a jowl fat deposit.

In some embodiments of the above methods, heating the tips of the microneedles comprises applying less than about 2.5 W of power to the microneedles. In some embodiments, heating the tips of the microneedles comprises applying about 100 mW to about 1000 mW of power (such as about 100 mW to about 500 mW of power, or about 500 mW to about 1000 mW of power) to the microneedles. In some embodiments, heating the tips of the microneedles comprises applying about 50 mW of power or less per microneedle. In some embodiments, heating the tips of the microneedles comprises applying about 1 mW to about 50 mW of power per microneedle. In some embodiments, the tips of the microneedles are heated for about 1 minute to about 20 minutes.

In some embodiments, the tips of the microneedles are heated to about 33° C. to about 60° C. In some embodiments, heating the tips of the microneedles comprises applying a direct current energy to the microneedles. In some embodiments, heating the tips of the microneedles comprises applying a radiofrequency energy to the microneedles.

In some embodiments of the above methods, the plurality of microneedles comprises about 3 microneedles to about 100 microneedles.

In some embodiments of the above methods, the microneedles comprise an insulated shaft, and wherein the tips of the microneedles are uninsulated.

In some embodiments of the above methods, the method comprises attaching a patch comprising the plurality of microneedles to skin above the fat deposit.

In some embodiments of the above methods, the method comprises placing a mask over the patch. In some embodiments, the method comprises wirelessly transferring energy from the mask to the patch, wherein the transferred energy heats the tips of the microneedles. In some embodiments, the method comprises controlling the heating of the tips of the microneedles using a computer system.

Further described herein is an apparatus for monitoring melting of a test substrate (e.g., a solid fat) using a device comprising a plurality of microneedles, comprising: a first surface and a second surface, the first surface comprising a transparent region, wherein the first surface and the second surface are parallel; a middle layer connecting the first surface to the second surface, the middle layer comprising a well containing the test substrate (e.g., the solid fat), wherein the well is visible through the transparent region of the first surface, and wherein the well is configured to receive tips of the plurality of microneedles. In some embodiments, the first surface or the second surface comprises glass or a thermally-resistant transparent material. In some embodiments, the middle layer comprises a polymeric foam or rubber. In some embodiments, the microneedles are configured to be heated using a power source. In some embodiments, the apparatus further comprises the device, wherein the tips of the plurality of microneedles are inserted in or are on the surface of the test substrate (e.g., the solid fat). In some embodiments, the transparent region comprises one or more graduated markers for quantitative analysis.

Also described herein is a method of monitoring melting of a test substrate (e.g., a solid fat), comprising applying energy to a plurality of microneedles inserted into or on the surface of the test substrate (e.g., the solid fat) using the apparatus described above; and monitoring melting of the test substrate (e.g. the solid fat). In some embodiments, monitoring the melting of the test substrate (e.g., the solid fat) at a plurality of different power levels. In some embodiments, monitoring the melting of the test substrate (e.g., solid fat) at a plurality of different time points. In some embodiments, the monitoring the melting of the test substrate (e.g., the solid fat) comprises determining a qualitative or a quantitative degree of melting.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A schematically illustrates a top view of a microneedle array in a disposable patch of a microneedle treatment system according to one embodiment of this disclosure.

FIG. 1B schematically illustrates the top view of the microneedle array substrate in the disposable patch in FIG. 1A.

FIG. 1C schematically illustrates the top view of a micro-coil in the disposable patch in FIG. 1A.

FIGS. 2A and 2B schematically illustrate a cross-section view of a microneedle array in a disposable patch of a microneedle treatment system according to one embodiment of this disclosure.

FIG. 3A schematically illustrates a cross-section view of a tip of a microneedle, with insulation, in a microneedle array according to one embodiment of this disclosure.

FIGS. 3B, 3C, 3D and 3E schematically illustrate some other examples of a tip of a microneedle with and without insulation, in a microneedle array according to some other embodiments of this disclosure.

FIG. 4A schematically illustrates a cross-section view of a hollow tip of a microneedle in a microneedle array according to one embodiment of this disclosure.

FIG. 4B schematically illustrates a cross-section view of a hollow tip of a microneedle in a microneedle array according to another embodiment of this disclosure.

FIG. 5A schematically illustrates a microneedle array on a rigid or flexible substrate.

FIG. 5B schematically illustrates a microneedle array including multiple sub-arrays on a flexible substrate to create a large-area semi-flexible array.

FIG. 6 schematically illustrates a disposable patch with a microneedle array applied under eyes in areas of fat deposits. The illustrated example shows reducing lower eyelid fat.

FIG. 7 schematically illustrates an overlying mask attached to a disposable patch to inductively power the disposable patch.

FIG. 8A schematically illustrates a disposable patch with a microneedle array applied to jowls.

FIG. 8B schematically illustrates a disposable patch with a microneedle array applied to nasolabial folds.

FIG. 9A schematically illustrates an upper face mask with coils to be placed over one or more disposable patches. The mask houses coils, which are flexible and can vary in substrate and size.

FIG. 9B schematically illustrates a lower face mask with a coil to be placed over one or more disposable patches. The mask houses coils, which are flexible and can vary in substrate and size.

FIG. 9C schematically illustrates a full face mask with coils to be placed over one or more disposable patches.

FIG. 10A schematically illustrates a method of applying one or more disposable patches in areas of fat deposits under the eyes to reduce lower eyelid fat.

FIG. 10B schematically illustrates a method of attaching a mask with coils to one or more disposable patches in areas of fat deposits under the eyes to reduce lower eyelid fat.

FIG. 11A schematically illustrates a method of attaching a mask with one or more coils to multiple disposable patches in areas of fat deposits in nasolabial folds.

FIG. 11B schematically illustrates a method of attaching a mask with one or more coils to multiple disposable patches in areas of fat deposits in the jowl region.

FIG. 12A schematically illustrates a method of attaching a full-face mask with coils to multiple disposable patches in multiple areas of fat deposits including under the eye, nasolabial folds and jowls.

FIG. 12B schematically illustrates a method of attaching a full-face mask with coils to multiple disposable patches in multiple areas of fat deposits including under the eye, nasolabial folds, jowls and under the chin area.

FIG. 13A shows a patch with an attached microneedle array directly attached to a power supply, which provide power to the microneedles to heat the microneedles.

FIG. 13B shows a mask configured to wirelessly transfer to two patches with attached microneedles through inductive power transfer. The mask includes an on-board rechargeable battery and a cable connector. A removable plug can plug into the cable connector and a wall socket to recharge the battery.

FIG. 14 is the cross section of an exemplary human face, showing the anatomy of facial tissues, in particular, the orbicularis oculi and the orbital septum that are positioned above the postseptal fat deposit.

FIG. 15 shows the degree of liquefaction of butter after applying 1000 mW of energy for 0 and 1 minute at room temperature, compared to untreated control butter.

FIG. 16A shows the degree of liquefaction of butter after applying 100 mW of energy for 10 minutes, 250 mW of energy for 5 minutes, or 500 mW of energy for 3 minutes as compared to untreated control butter.

FIG. 16B shows the degree of liquefaction of butter after applying 50 mW of energy for 10 minutes, 100 mW of energy for 10 minutes, 250 mW of energy for 5 minutes, or 500 mW of energy for 3 minutes as compared to untreated control butter.

FIG. 17 shows the degree of liquefaction of chicken fat over time (0-15 min) with application 1000 mW of energy as compared to untreated control chicken fat.

FIG. 18 shows the degree of liquefaction of chicken fat after applying 250 mW of energy, 350 mW of energy, or 500 mW of energy for 5 minutes as compared to untreated control chicken fat.

FIG. 19A shows two patches of the system attached to a power supply. The power supply includes a display, a power button, a start button, and a stop button.

FIG. 19B shows two patches of the system described herein attached to skin underneath the eyes of a subject. The patches each include a microneedle array, and the microneedles are inserted into the subcutaneous fat. Wires extend from the patches, which connect to a power supply.

FIG. 20A illustrates top, front, and side views of an exemplary embodiment of a dome-shaped patch.

FIG. 20B illustrates a cross-section of the patch illustrated in FIG. 20A, showing a plurality of microneedles within the cavity of the dome-shaped body.

FIG. 20C illustrates an exploded view of the patch with the dome-shaped body of the patch illustrated in FIG. 20A.

FIG. 21A shows a cross-section of another example of the dome-shaped patch.

FIG. 21B shows an underneath view of the patch illustrated in FIG. 21A.

FIG. 21C shows an exploded view of the patch illustrated in FIG. 21A.

FIG. 22A shows a bottom and side view of an exemplary patch.

FIG. 22B shows a perspective view of the patch shown in FIG. 22A.

FIG. 22C shows an exploded view of the patch illustrated in FIG. 22A.

### DETAILED DESCRIPTION

The present disclosure now will be described in detail with reference to the accompanying figures. This disclosure may be embodied in many different forms and should not be construed as limited to the example embodiments discussed herein.

Described herein are microneedle treatment systems for reduction of unwanted and protruding pockets of fat that exist just below the surface of the skin, or in close proximity to the skin, using pain-free and safe methods. Some examples of these areas include, but are not limited to, the fat pads below the eye (“baggy or puffy eye” appearance, blepharochalasis, dermatochalasis), along the jaw line (jowls), along the cheek smile lines (nasolabial folds), and below the chin (“double-chin” or sub-genial fat). The systems and methods are also able to address targeted deep layers of the skin. The systems and methods will enable both in-office and home treatment of conditions that currently can only be treated in a physician's office.

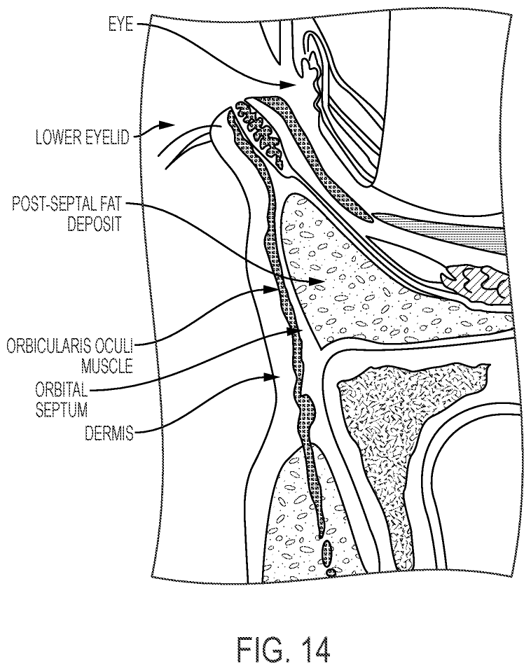

Anti-aging treatments described herein involve removing or reducing subcutaneous fat deposits, which can result in tightening skin on the body. Certain embodiments target subcutaneous fats in the facial region, such as the periorbital postseptal or preseptal fat deposits (more commonly known as fat bags around) which may be around, above, or below the eye. For example, the targeted fat deposit may be a postseptal fat deposit or a preseptal fat deposit on the above the eye, or a postseptal fat deposit or a preseptal fat deposit below the eye. In some embodiments, the subcutaneous fat deposit a sub-orbicularis oculi fat (SOOF) fat deposit or a retro-orbicularis oculi fat (ROOF) deposit. Microneedles penetrate through the dermal layer, and the tips of the microneedles are used to apply energy to, and melt, the subcutaneous fat deposits under the dermal layer. Certain subcutaneous facial fat deposits are located underneath a thin muscle layer and/or other membrane tissue. For example, for the lower eyelid, periorbital postseptal fat deposits are located underneath the orbicularis oculi muscle and the orbital septum (see FIG. 14). Periorbital preseptal fat deposits are located between the muscle layer and the orbital septum. The microneedles of devices described herein for reducing postseptal fat deposits penetrate the dermal layer as well as the muscle layer and/or membrane layer to reach the subcutaneous fat deposits, such as periorbital preseptal facial fat deposits or periorbital postseptal facial fat deposits.

Subcutaneous fats are found beneath the skin, as opposed to visceral fats, which are found in the peritoneal cavity. Subcutaneous fats targeted by the present invention include facial fats, as well as non-visceral fats on other parts of the body, such as arms, elbows, shoulders, abdomen, or legs. Subcutaneous fats exist in various facial regions, and accumulation of these subcutaneous fats can cause loosening of skin and a puffy or aged appearance, such as when postseptal or preseptal fats give rise to engorged “eye fat bags” below the eyelid. Therefore, the elimination or reduction of these and other subcutaneous fats can improve skin tightening and prove useful in anti-ageing therapy. Certain targeted subcutaneous fats are about 8 mm or less below the skin surface, such as about 1 mm to about 2 mm, about 2 mm to about 3 mm, about 3 mm to about 4 mm, 4 mm to about 5 mm, about 5 mm to about 6 mm, about 6 mm to about 7 mm, or about 7 mm to about 8 mm below the skin surface.

Previous microneedle treatment systems have been designed to use shorter needles or adjustable length needles to apply energy to the dermal layer (rather than tissue below the dermal layer), and often employ moving needle parts and a greater range and higher energy to treat the dermal layers. This increases the risk of burns and other injuries to the overlaying tissues, potentially causing inflammation or even necrosis, and significantly hampering any anti-aging treatments. The microneedle treatment system described herein is a safe and effective system for reducing subcutaneous (i.e., non-visceral) fat deposits. In certain embodiments, the system includes a patch that includes a microneedle array and a power supply. The microneedle array includes fixed-length microneedles, which allow for precise placement of the microneedle tip in the targeted subcutaneous fat deposit.

Other devices with variable microneedle lengths may have imprecise microneedle tip placement, which would cause heating in an undesired location such as within the dermal layers. Applying heat to the epidermal and dermal layer rather than the targeted fat deposit can result in ineffective fat treatment and/or risk injury to fragile dermal structures. Additionally, the power supply of the device is configured to provide a low energy power that is effective for heating the microneedle tips to melt fat when the microneedle tips are precisely placed in or on the surface of the targeted fat deposit. The low power device enhances safety compared to other, more powerful devices, which allows the system to be used for at-home treatment without risk of injury. For example, the power supply may be configured to heat the microneedles using less than about 2.5 W of power. In some embodiments, the power supply is configured to heat the microneedles using about 50 mW of power per microneedle, or less.

In one aspect of the present invention, there is a microneedle treatment system, comprising a microneedle array attached to a patch, the microneedle array comprising plurality of fixed-length microneedles comprising an insulated shaft and an uninsulated tip; and a power supply configured to heat the plurality of microneedles using less than about 2.5 W of power (such as about 100 mW to about 1000 mW of power, about 100 mW to about 500 mW of power, or about 500 mW to about 1000 mW of power). In some embodiments, there is a microneedle treatment system, comprising: a microneedle array attached to a patch, the microneedle array comprising a plurality of fixed-length microneedles, the microneedles comprising an insulated shaft and an uninsulated tip; and a power supply configured to heat the plurality of microneedles using about 50 mW of power or less per microneedle (such as about 1 mW to about 50 mW of power per microneedle) in the microneedle array. The system may be used, for example, to tighten skin or reduce a subcutaneous fat deposit, including facial fat deposits and other subcutaneous fat deposits in the body. The patch may be attached to the skin above the fat deposit to be treated, with the tips of microneedles inserted into the targeted fat deposit or positioned on the surface of the targeted fat deposit. In certain embodiments of the invention, a mask (which may be a reusable mask) is used with the patch, and can provide power to the patch to heat the microneedles.

Some of the advantages conferred by the current invention include a safer energy output profile to reduce risks of burns or other injuries to the subject receiving treatment. In some embodiments, the power supply can be configured to heat the microneedles using less than about 2.5 W of power, such as about 50 mW to about 1 W, about 1 W to about 1.5 W, about 1.5 W to about 2 W, or about 2 W to less than about 2.5 W of power. In certain examples, it is adequate for the power supply to heat the microneedles can using lower ranges of power, such as from about 100 mW to about 1000 mW, about 100 mW to about 500 mW of power, or about 500 mW to about 1000 mW of power. For example, in some embodiments power supply can be configured to heat the microneedles using about 50 mW to about 100 mW, about 100 mW to about 200 mW, about 200 mW to about 300 mW, about 300 mW to about 400 mW, about 400 mW to about 500 mW of power, about 500 mW to about 600 mW, about 600 mW to about 700 mW, about 700 mW to about 800 mW, about 800 mW to about 900 mW, about 900 mW to about 1000 mW of power.

The system can be adapted to include a desired number of microneedles, which are heated using low power. For example, in some embodiments, the power supply is configured to heat the plurality of microneedles in the array using about 50 mW of power or less per microneedle, such as about 1 mW to about 50 mW of power per microneedle. In some embodiments, the power supply is configured to heat the plurality of microneedles using about 1 mW to about 5 mW, about 5 mW to about 10 mW, about 10 mW to about 25 mW, or about 25 mW to about 50 mW of power per microneedle in the array.

Overheating of tissue can lead to discomfort, burns or even permanent damage. A control mechanism built into the system (such as the patch or, if included in the system, the mask) to limit the maximum energy delivered and/or regulate the temperature of the microneedle can reduce risks of overheating. In some embodiments according to any one of the devices described above, the patch, the power source, and/or the mask comprise a pre-set limit of maximum energy delivered and/or temperature regulator configured to suspend heating of the microneedles if the temperature goes above a predetermined threshold.

Some aspects of the current invention can include features for securing the patch onto the targeted treatment area, such as a skin-safe (and preferably, thermal resistant) adhesive or a masked configured to be placed over the patch to hold the patch in place. For example, the system can include a mask comprising the power supply, wherein the mask is configured to be placed over the patch. In some embodiments, the mask is configured to be placed over, around, or below an eye of a human subject, and over the patch. In some embodiments, the mask can cover the upper portion of the face (upper face mask), the lower portion of the face (lower face mask), the left side of the face, the right side of the face, or the entire face (full face mask). In some embodiments, the patch can be placed over one or more of the lower eyelid, the nasolabial fold, or the jowl regions.

Other similar microneedle devices often require the subject or a physician to provide physical support for the device to properly contact the treatment area. One advantage presented by the microneedle device in the current application is the convenience of hands-free operation. In some embodiments, the device is a hands-free device. In some non-limiting examples, a hands-free device can comprise features that allow the system to be attached to the targeted treatment area without external support. In some embodiments according to any one of the devices described above, the patch comprises an adhesive. In some embodiments, the adhesive provides non-permanent adhesion.

The length of the microneedles depends on the targeted subcutaneous fat deposit. In a non-limiting example, the microneedles can be about 2 mm to about 5 mm in length. In some embodiments, the treatment device comprises microneedles of about 1.5 mm to about 2 mm, about 2 mm to about 2.5 mm, about 2.5 mm to about 3 mm, about 3 mm to about 3.5 mm, about 3.5 mm to about 4 mm, about 4 mm to about 4.5 mm, or about 4.5 mm to about 5 mm in length. In one example, such as when the device is used to reduce periorbital postseptal fat, the microneedles are about 3 mm to about 4 mm in length, such as about 3.5 mm.

In some embodiments, the shaft of the microneedles is about 500 μm in diameter or less, for example about 50 μm to about 60 μm, about 60 μm to about 80 μm, about 80 μm to about 100 μm, about 100 μm to about 120 μm, about 120 μm to about 140 μm, about 140 μm to about 160 μm, about 160 μm to about 180 μm, about 180 μm to about 200 μm in diameter, about 200 μm to about 250 μm in diameter, about 250 μm to about 300 μm in diameter, about 300 μm to about 400 μm in diameter, or about 400 μm to about 500 μm in diameter.

The microneedle array on the patch can include a plurality of microneedles in any suitable configuration, such as a square, rectangle, triangle, circle, oval, or crescent shaped. In some embodiments, the microneedle array includes between 2 and about 100 microneedles, such as 2 to about 10 microneedles, about 10 to about 20 microneedles, about 10 to about 25 microneedles, about 25 to about 50 microneedles, or about 50 to about 100 microneedles.

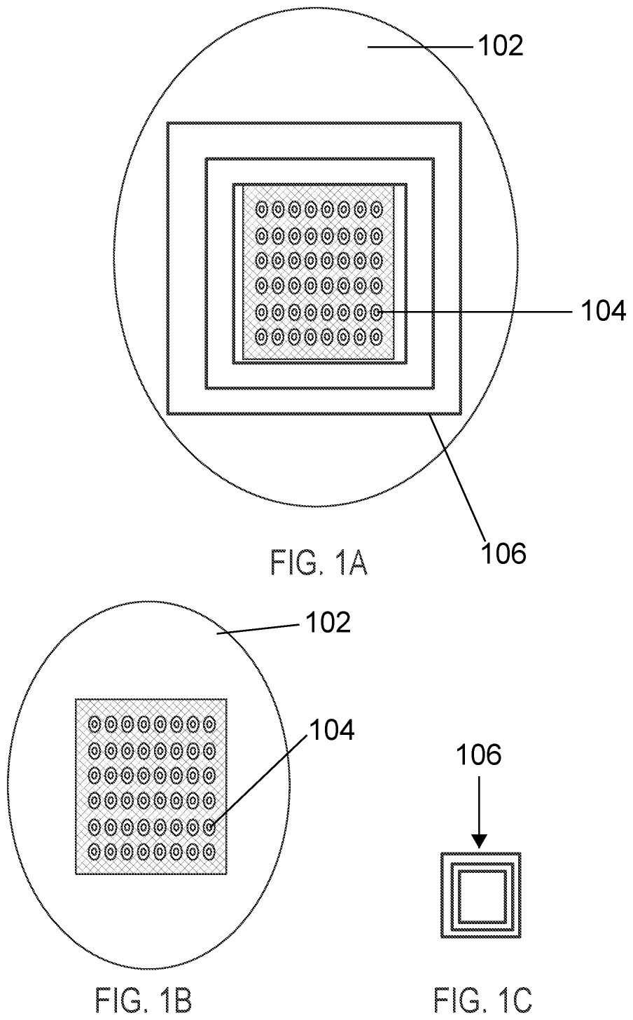

FIGS. 1A-1C schematically illustrate a top view of a microneedle array in a disposable patch of a microneedle treatment system, possibly including a micro-coil and/or antenna in the disposable patch, in one embodiment. FIG. 1B shows the patch 102, for which the substrate, shape, or size can vary; and needles 104, for which the substrate, coating, size, number and spacing can vary. FIG. 1C shows a microcoil and/or antenna 106 (circuit attach points not shown). The microneedles can be sized to make their insertion pain-free and imperceptible to a user. Microneedles can be smaller than 200 μm in diameter and up to 3500 μm in length in order to not cause pain upon insertion in the skin. For example, a diameter of approximately 100 μm is too small to be sensed by human nerves. The solid microneedles can be anywhere from 50 to 3500 μm in length. The length of the needle and shape of the microneedle array and patch can be designed to correspond to the targeted anatomic area and skin or fat layer to be addressed. This microneedle treatment system can penetrate to the desired depth (deep dermis or deeper than skin) to deliver energy through the tips of the microneedles to reduce the targeted fat layer, or direct energy in a highly-focused manner to the targeted skin layer, thus minimizing the energy being directed to the surrounding off-target areas.

The disposable patch can include a plurality of micro-needles organized in an array (row and column) format on a thin, flexible substrate. The microneedles protrude from a flexible substrate which can be temporarily adhered to the patient's body on the area needing treatment. The microneedles in the array can be electrically interconnected. For example, the disposable patch can include the microneedles discussed in U.S. Pat. Nos. 7,785,459, 7,846,488, 7,785,301, 7,627,938, and 7,412,767. The disposable patch can also include a micro-coil to be used as an antenna for communications as well as for inductive power transfer.

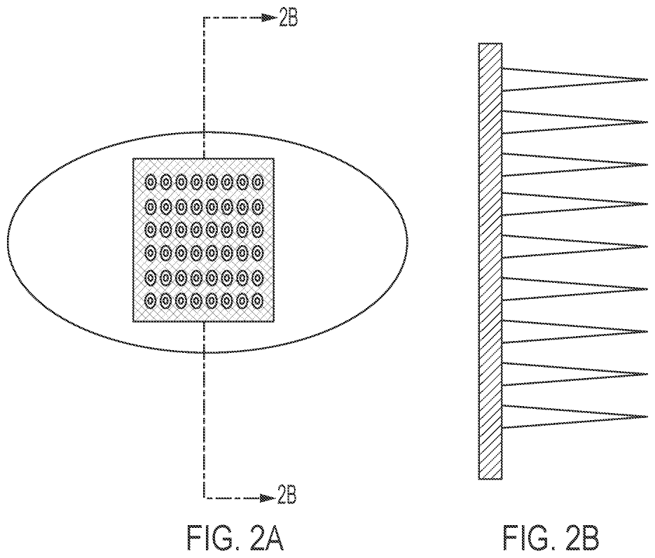

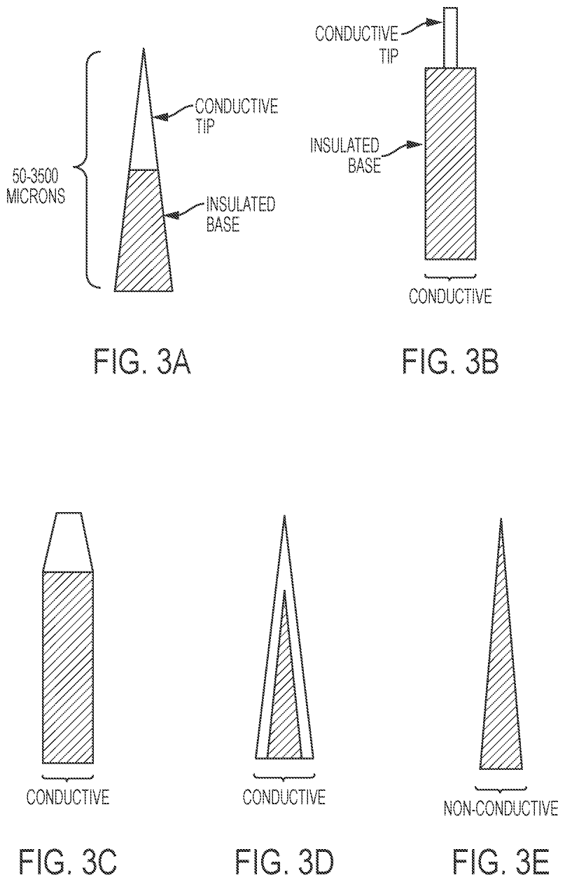

FIGS. 2A and 2B schematically illustrate a cross-section view of a microneedle array of a microneedle treatment system in one embodiment. FIG. 2B is a cross-section along the line “2B-2B” shown in FIG. 2A. FIG. 3A schematically illustrates a cross-section view of a tip of a microneedle in one embodiment. The microneedle can have a conductive tip and an insulated base. FIGS. 3B-3E schematically illustrate some other examples of a tip of a microneedle in a microneedle array in some other embodiments. For example, the tip of the microneedle can have different shapes or even be non-conductive. For the exemplary needles indicated in FIG. 3A-3E, the needle height (i.e., the depth of needle range) should be 0.05 mm to 3.5 mm.

The microneedles include a thermally conducive material and/or an electrically conductive material. Optionally, the microneedles can include a non-conductive core and a thermally or electrically conductive coat, or a thermally or electrically conductive core and a non-conductive coat. In some embodiments, the microneedles may be made from any material that is thermally conductive and biologically compatible for subcutaneous use, such as single-crystal silicon, stainless steel, titanium, gold, platinum, or non-biocompatible materials that have been coated with biocompatible substances.

The microneedles may be fabricated by methods such as: semiconductor and MEMS processes, employing micron-scale photolithographic patterning, physical vapor deposition, chemical vapor deposition, thermal oxidation, plasma etch and/or chemical etch; traditional metal machining processes such as cutting, grinding and electrical discharge machining (EDM); direct deposition or 3D printing of thermally conductive substances; electroplating; chemical milling; molding. The microneedles can be bare or insulated (except for the tips) by various means including but not limited to: vapor-phase coating, layering with non-conductive substrate, electroporation, and materials known to have beneficial effects on scars, such as silicone polymers.

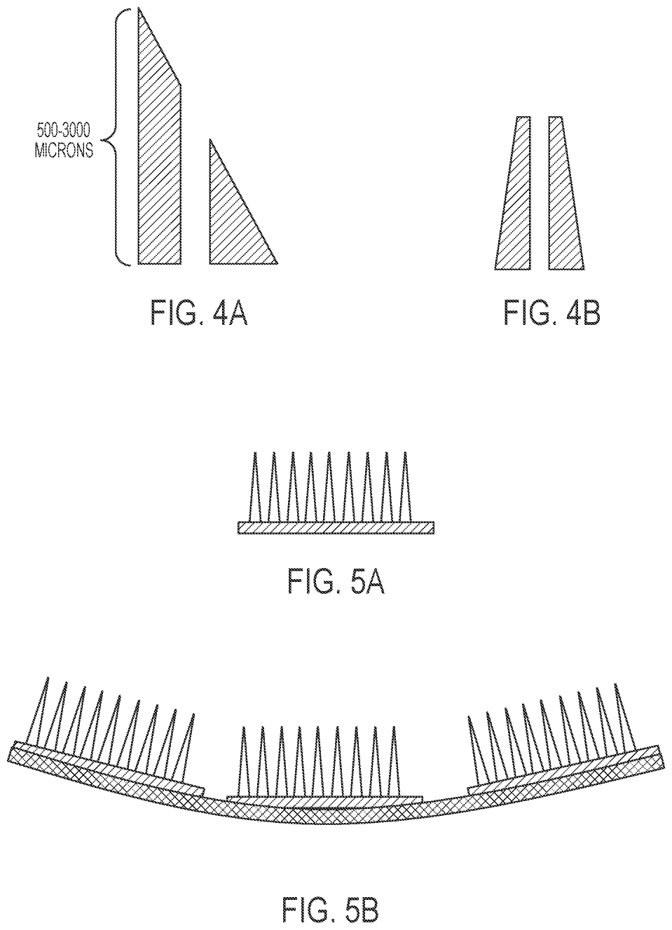

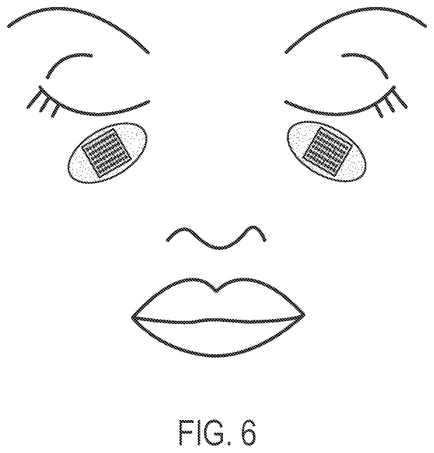

Microneedles can be made by various methods known to those skilled in the art of micromachining. Microneedles may be made with or without hollow cores. FIG. 4A schematically illustrates a cross-section view of a hollow tip of a microneedle in one embodiment. The microneedle shown in FIG. 4A has a sharp tip and is between 500 microns to 3000 microns in length FIG. 4B schematically illustrates a cross-section view of a hollow tip of a microneedle in another embodiment. The microneedle illustrated in FIG. 4B has a blunt tip.

Microneedles can be arrayed in many different configurations (rows and columns, hexagonal-packed) and spacing pitches. For example, microneedles may be formed by electroplating metals onto a substrate and building up metal in a micromold. Using another fabrication method, the microneedles may be etched from a rigid material such as glass or silicon and then a thin layer of metal may be deposited over the surface of the needle tips to form a conductive surface.

FIG. 5A schematically illustrates a microneedle array on a rigid or flexible substrate. The microneedle array could be disposed onto a rigid or flexible substrate as shown in FIG. 5A. If a very small area of skin is to be treated, then a microneedle array on a rigid substrate might be sufficient.

FIG. 5B schematically illustrates a disposable patch including multiple microneedle sub-arrays on a flexible substrate to create a large-area semi-flexible array. If a large area of the body is to be treated, then the microneedle array may have to be able to conform to the body surface. It is advantageous to apply the disposable patch including multiple microneedle sub-arrays to create a large-area flexible or semi-flexible array. As shown in FIG. 5B, multiple microneedle sub-arrays can be disposed onto a series of rigid substrates that are tiled to form a semi-flexible substrate to cover a large treatment area.

The disposable patch can vary in size, and in some embodiments are as small as approximately 1 cm×1 cm and is shaped to adhere comfortably to the designated treatment area. Patch shapes such as ellipse, crescent, semi-circle, teardrop, triangle, rectangle, circular, oblong, oval, square etc. are possible. Multiple patches can be applied to various locations under the eyes, according to a person's needs, face shape, and size. The patch of the device in the current application can be designed to conform to the contours of the targeted treatment area for comfort and proper fitting.

In some embodiments, the patch has a dome shaped-body, with microneedles protruding from the top of the dome toward a base within the cavity formed by the dome. The bases of the microneedles (i.e., the portion of the microneedles distal from the tip of the microneedles) are attached to the upper portion of the dome shape along the inner surface. The base of the dome can be, for example, a circle or an oval. The bottom of the base can optionally include an adhesive, which allows the patch to be fixed to the skin. The base of the dome may include a lip (which can protrude toward the center of the base or away from the center of the base, or both), which provides an additional contact surface for the skin. The adhesive can be disposed on the bottom portion of the lip. The dome-shaped can be formed from a flexible material (for example a flexible plastic (e.g., polyethylene, polypropylene, polyvinyl chloride, nylon, or polyester) or silicone rubber), and can be configured from the dome shape to a substantially flat configuration. When the dome-shape patch is placed on the skin, the top of the dome can be pressed down toward the skin, thereby configuring the body in the substantially flat configuration and inserting the microneedles into position. When the dome-shaped patch is configured into the substantially flat configuration, the base of the dome (which preferably includes an adhesive to attach the patch to the skin) stretches the skin. Stretching the skin while simultaneously inserting the microneedles allows for decreased pain as the needles are inserted (compared to un-stretched skin).

Optionally, the patch with the dome-shaped body includes a needle guard proximal to the base of the dome. The needle guard caps the cavity formed by the dome, and includes a plurality of openings (e.g., holes or slits) configured to allow passage of the tips of the microneedles. Therefore, when the dome-shaped patch is configured in the substantially flat configuration, a portion of the microneedles (such as the tips or a portion of the shaft proximal to the tips) protrude through the holes of the needle guard and into the skin and/or subdermal fat deposit.

The dome-shaped patch in the substantially flat configuration can be removed, and returns to its original three-dimensional dome shape and the microneedles are auto-returned into the dome cavity. The dome-configuration of the patch allows it to fold in on itself and serve as self-contained protective disposal unit for the microneedles. If the patch includes the needle guard, the microneedle tips preferably retract into the cavity past the needle guard.

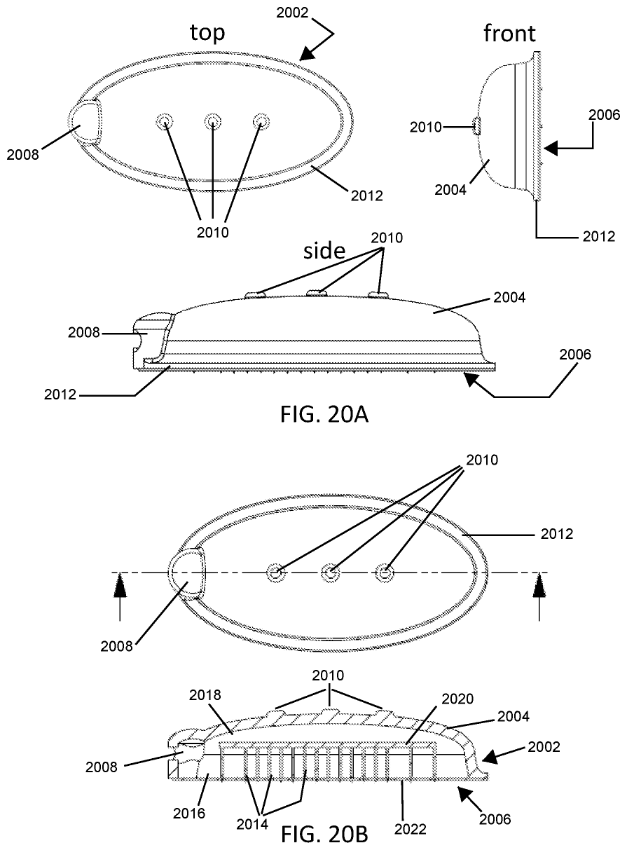

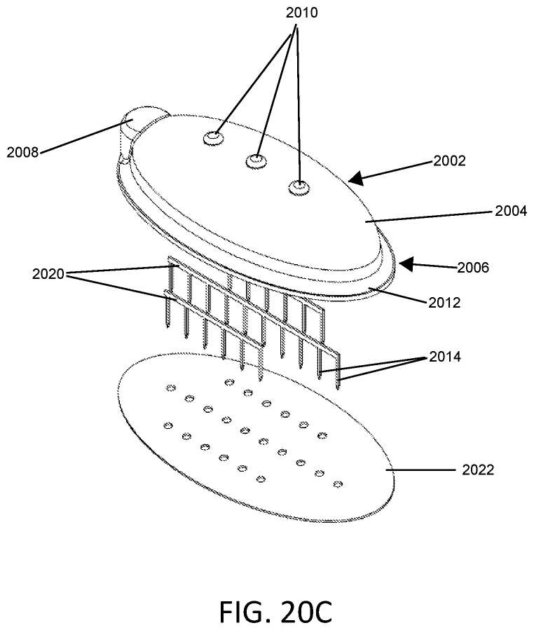

FIG. 20A illustrates top, front, and side views of an exemplary embodiment of a dome-shaped patch. The patch includes a flexible dome-shaped body 2002, with an upper portion 2004 and a base 2006. In the illustrated embodiment, the base 2006 of the dome is oval shaped. The patch can include a wire port 2008, which can link the plurality of microneedles to a power supply, although in some embodiments the power supply wirelessly provides energy to the patch to heat the microneedles. The illustrated example includes optional protruding members 2010 on the top of the outer surface of the dome shaped body 2002. The protruding members 2010 provide a tactile indication to the user as to where to apply pressure to the patch to change the form of the body of the patch from a dome shape into a substantially flat configuration. The base 2006 of the body includes a lip 2012. The bottom portion base 2006 (e.g., the lip 2012) contacts the skin of the subject, and may include an adhesive. FIG. 20B illustrates a cross-section of the patch, showing a plurality of microneedles 2014 within the cavity 2016 of the dome-shaped body 2002. The microneedles 2014 are attached to the inner surface 2018 of the body 2002, and are optionally stabilized by one or more cross-bars 2020 that connects at least a portion of the bases of the microneedles (that is, the portion of the microneedles relative to the tip), and the one or more cross-bars can be directly or indirectly attached to the inner surface of the body. The illustrated embodiment further includes the optional needle guard 2022. The profile of the needle guard takes the shape of the base and encloses the cavity 2016. The needle guard includes a plurality of holes, which allows passage of the microneedles 2014 when the body is configured in the substantially flat configuration from the dome-shaped configuration. The tips of the microneedles may protrude through the needle guard 2022 even when the body is in the dome-shaped configuration; however the needle guard 2022 can still protect the microneedles from damage and can hide the visual appearance of the microneedles. FIG. 20C illustrates an exploded view of the patch with the dome-shaped body.

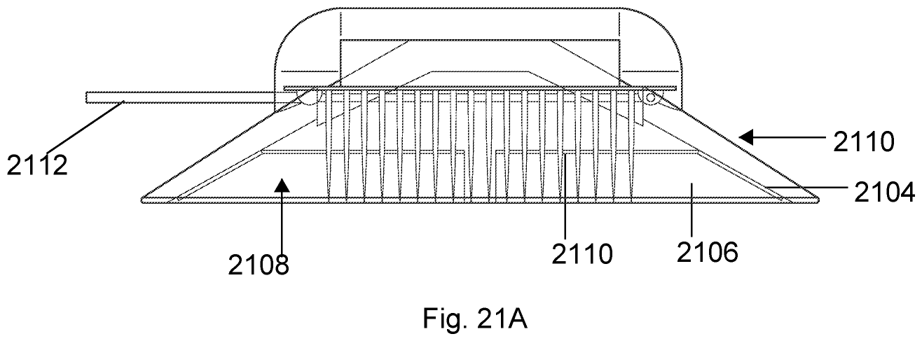

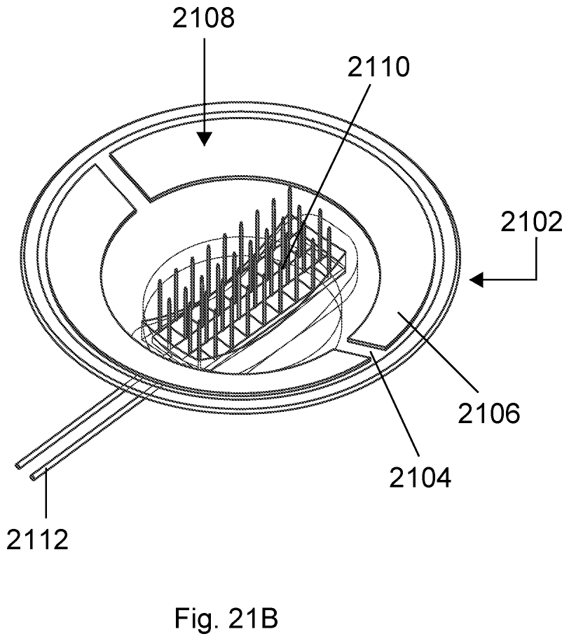

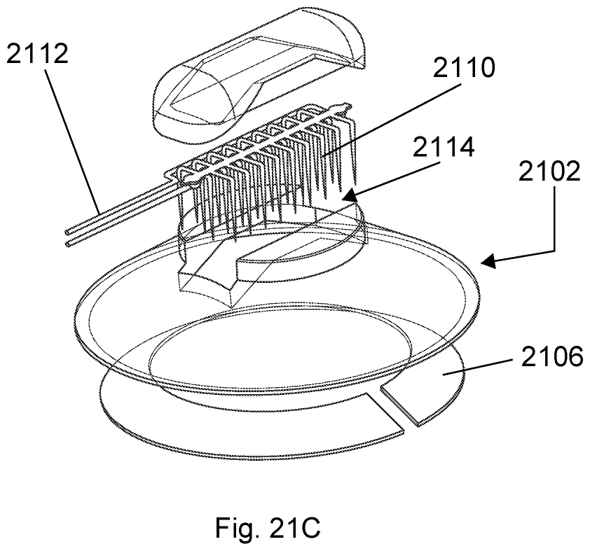

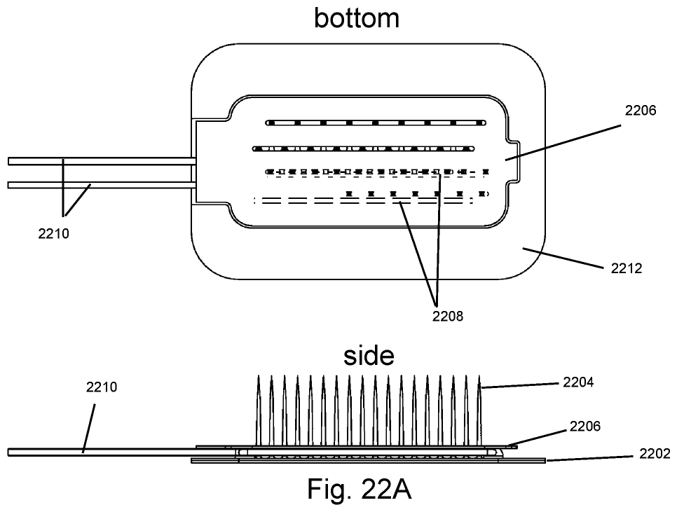

FIGS. 21A-21C illustrate another embodiment of a patch with a dome-shaped body 2102 that can be changed into a substantially flat configuration. FIG. 21A shows a cross-section of the patch, while FIG. 21B shows an underneath view and FIG. 21C shows an exploded view of the patch. The body 2102 is formed from flexible material, and can function as a suction cup when the body 2102 is attached to the skin. The inner surface 2104 of the body 2102 can include an adhesive 2106, which can bond to the surface of the skin of the subject. The cavity 2108 of the body 2102 houses a plurality of microneedles 2110. The bases (i.e., the portion distal from the tips) of the microneedles 2110 are connected to wires 2112, which are connected to a power supply (although the power could be supplied to the microneedles through wireless energy transfer, as described herein), to provide energy to the microneedles to heat the microneedles. The top of the body 2102 can include an opening 2114, which allows the microneedles to pass through into the cavity 2108 while the wires 2112 remain outside of the cavity 2108. A cap 2116 (e.g., an epoxy cap) can cover the bases of the microneedles 2110, which holds the microneedles in position. When patch is reconfigured into a substantially flat configuration, the body 2102, held to the skin by the adhesive 2106, stretches the skin and the microneedles a repositioned such that a portion of the microneedle from within the cavity is repositioned below the base. This allows the microneedles to be inserted into the skin of the subject when the body is reconfigured into the substantially flat configuration.

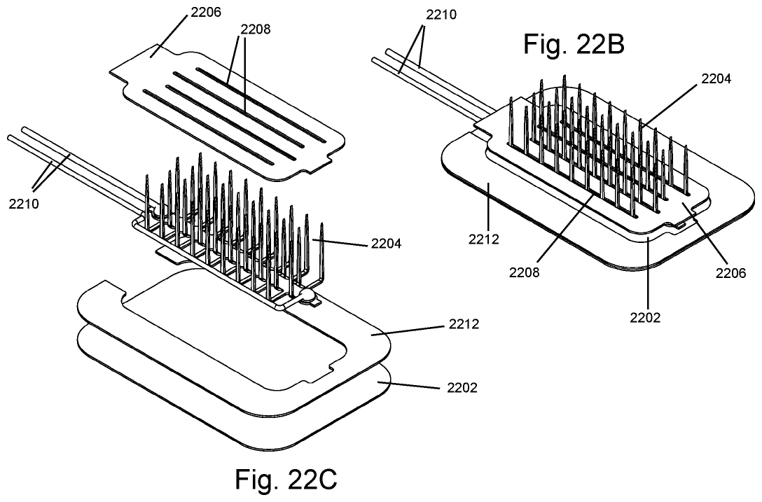

FIGS. 22A-22C illustrate an embodiment of the patch with a substantially flat body 2202. FIG. 22A shows a bottom and side view of the patch, while FIG. 22B shows a perspective view and FIG. 22C shows an exploded view. A microneedle array 2204 that include a plurality of microneedles is attached to a bottom surface of the body 2202 such that the tips of the microneedles are directed away from the body 2202. The microneedle array 2204 is attached to wires 2210, which are configured to provide energy to the microneedles to heat the microneedles. In some embodiments, the wires 2210 are attached to a power supply, and in some embodiments, the power supply wirelessly provides energy to the wires to heat the microneedles. A needle guard 2206 that includes a plurality of slits 2208 is attached to the body and holds the microneedle array 2204 in place. In the illustrated embodiment, the needle guard 2206 protects damage to the microneedles by securing the bases of the microneedles to the body. However, in this embodiment the needle guard 2206 does not substantially protect the shafts or tips of the microneedles, and a secondary needle guard may be used. An adhesive 2212 can also be attached to the bottom surface of the body 2202, which bonds the patch to the skin of the subject when the patch is in use. The adhesive can surround the needle guard on an exposed portion of the body 2202. In some embodiments, the needle guard also include an adhesive on the exposed portion.

To reduce the risk of unintended energy transfer to tissues surrounding the targeted treatment area, the microneedle can have a conductive tip and an insulated shaft, thereby exposing a tip of the microneedle for energy conduction. The conductive tip can transfer energy to the applied tissues while the insulated shaft will prevent an undesired transfer of energy to surrounding tissues. The length of the uninsulated tip can be adjusted according to the applications for melting facial fats in different targeted areas. In some embodiments, the uninsulated tip is about 0.1 mm to about 1.0 mm in length. In some further examples, the uninsulated tip can be any one of about 0.1 mm to about 0.2 mm, about 0.2 mm to about 0.3 mm, about 0.3 mm to about 0.4 mm, 0.4 mm to about 0.5 mm, about 0.5 mm to about 0.6 mm, about 0.6 mm to about 0.7 mm, about 0.7 mm to about 0.8 mm, about 0.8 mm to about 0.9 mm, about 0.9 mm to about 1.0 mm, about 1.0 mm to about 1.1 mm, or about 1.1 mm to about 1.2 mm in length. The insulated portion of the microneedle is coated with a suitable insulating material, which may be thermally and/or electrically insulating. Exemplary insulating materials include parylene, glass, and polytetrafluoroethylene.

The microneedle treatment system described herein can include a disposable patch and a reusable overlying mask. The disposable patch can include a microneedle array including a plurality of microneedles. The overlying mask can be configured to be placed directly over the disposable patch. The mask can include a drive circuitry configured to deliver energy into the microneedle array, one or more sensors configured for localized sensing, and a telemetry uplink to a smartphone, computer or the internet. The overlying mask can be powered by an electric control unit via an electrical wall plug cable or directly by an onboard rechargeable battery. The microneedle treatment systems use MEMS or MEMS-like technology to delivery energy to reduce fat deposits directly under or in close proximity to the skin, in order to thicken and tighten dermis to treat skin laxity and wrinkles, to improve skin scars, and to treat other skin problems.

The microneedle treatment system described herein delivers a thermal output with enhanced safety for the subject. As opposed to using high heat to melting fats, the device described herein operates under a safer range of temperatures. In some embodiments according to any one of the devices described above, the power supply in the microneedle device is configured to heat the tips of the microneedles from about 33° C. to about 65° C. In further embodiments, the power supply in the microneedle device is configured to heat the tips of the microneedles from any one of about 30° C. to about 33° C., about 33° C. to about 35° C., about 35° C. to about 40° C., about 40° C. to about 45° C., about 45° C. to about 50° C., about 50° C. to about 55° C., about 55° C. to about 60° C., or about 60° C. to about 65° C.

Energy provided by the power supply to heat the microneedles may be a direct current (DC) energy or a radiofrequency (RF) energy. For example, a direct current may be applied to the microneedles, which cause the microneedles to heat. Thermal insulation surrounding the microneedle shaft prevents damage to the dermal layers and allows the heat to radiate from the non-insulated microneedle tips implanted in the targeted subcutaneous fat deposit. Radiofrequency energy can also be used to heat the microneedles, which allows an electrical current to pass between microneedles. By electrically insulating the shafts of the microneedles, the radiofrequency energy is narrowly applied to the tips of the microneedles, thereby preventing damage to the dermal layer.

The power supply may be connected directly to the microneedles through electronic circuitry (i.e., connected to the microneedles through one or more wires), or may be wirelessly connected to the microneedle array. For example, in some embodiments, the patch is directly connected to the power supply, which supplies power to the microneedles. In some embodiments, the power supply is directly attached to another device (such as a mask), which wirelessly transfers power to the patch to supply power to the microneedles. The power supply can include a battery (which may be a rechargeable battery), or can include an electrical plug (which may be permanently attached or removable) that can be used with a wall socket.

Power can be wirelessly transferred to the patch to power the microneedle array through inductive power transfer, radiofrequency power transfer, near-field power transfer, non-radiative power transfer, radiative power transfer, or any other suitable method. For example, the system can include a mask that includes a power supply, and the mask and the patch can be configured to wireless transmit power from the mask to the patch to heat the microneedles. In an example of a device configured for wireless power transfer, the patch can include a first antenna electrically connected to the microneedle array, and the power supply includes a second antenna. The power supply can be in a housing (such as a mask) separate from the patch comprising the microneedles. To transfer power from the housing to the microneedle array, the power supply transfers power to the patch through inductive power transfer. In some embodiments, the power supply includes a battery, and can be a wireless device.

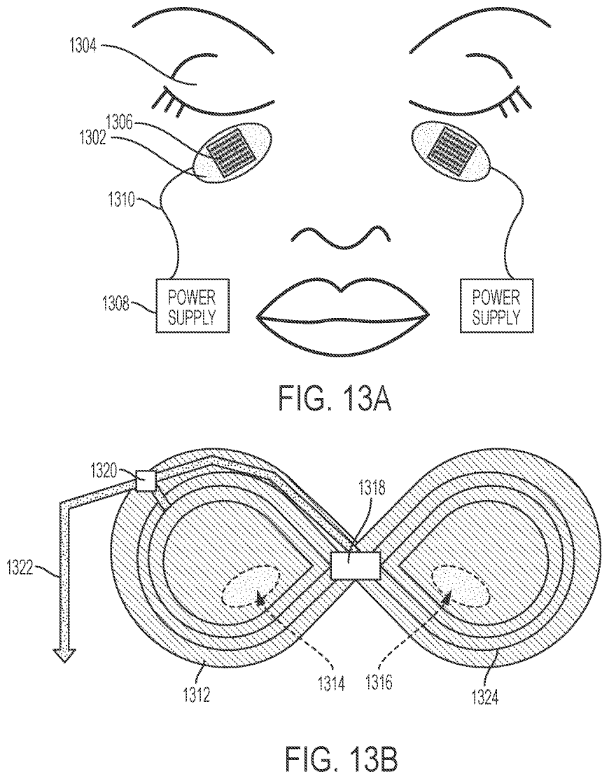

FIG. 13A shows a patch with an attached microneedle array directly attached to a power supply, which provide power to the microneedles to heat the microneedles. In the illustrated embodiment, patch 1302 includes an adhesive surface to attach the patch underneath the eye 1304 of the subject. The patch 1302 includes a microneedle array 1306, and the needles within the microneedle array are electrically connected through a wire. The wire is directly connected to a power supply 1308 through a wire 1310. Although the power supply 1302 is shown in FIG. 13A as connected to the patch 1302 through a wire 1310, it is contemplated that the power supply 1302 can be an onboard power supply that is positioned on or within the patch 1302. Further, although FIG. 13A illustrates two patches each having their own power supply, it is contemplated that a single power supply can be shared between two or more patches.

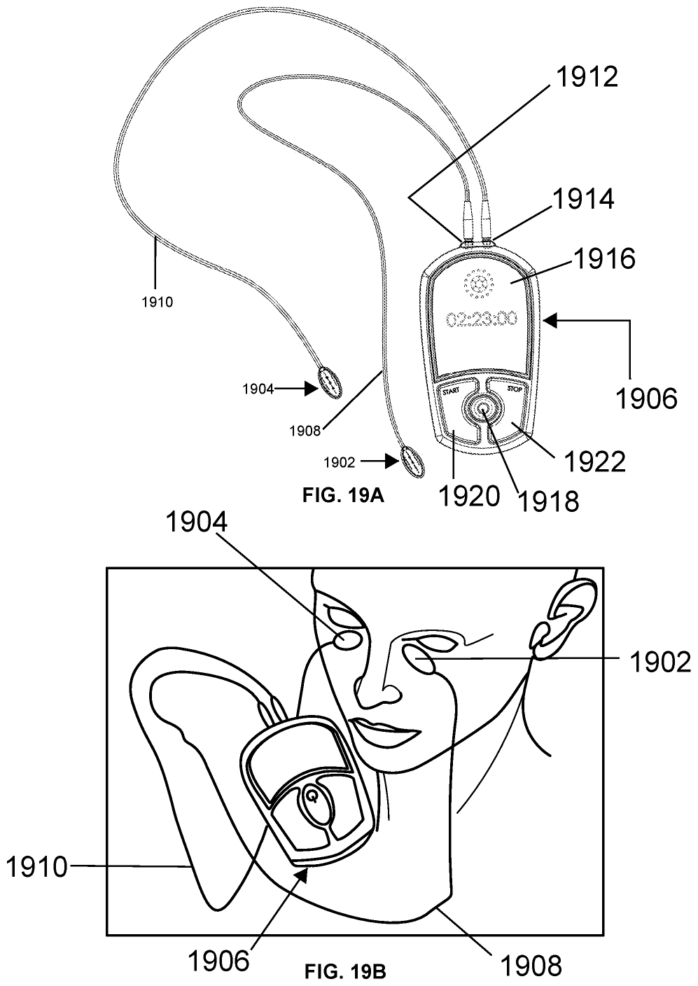

FIG. 19A illustrates a microneedle treatment system that includes two patches (1902 and 1904) attached to a power supply 1906. In the illustrated embodiment, patch 1902 is connected to the power supply 1906 through a first wire 1908, and patch 1904 is connected to the power supply 1906 through a second wire 1910. Wire 1908 connects to the power supply 1906 at port 1912, and wire 1910 connects to power supply 1906 at port 1914, although it is conceived that the wires can connect to the power supply through a single port. The power supply 1906 includes a display 1916. The display 1916 can present, for example, status relating to the power supply (such as battery level, whether the power supply is on or off, an amount of power being supplied to the patches, and mount of time that power has been supplied to the patches since being turned on, or an amount of time remaining for the power to be supplied to the patches) or a status relating to the patches (such as microneedle temperature). As shown in FIG. 19B, the patches 1902 and 1904 can be attached to the skin of the subject, such as underneath the eye of the subject. The microneedle array on the bottom surface of patch 1902 is inserted into the septal fat, and the power supply 1906 is turned on to supply power to the patch 1902. Patch 1904 also include a microneedle array, which can be inserted into a subcutaneous fat deposit at a different location. The illustrated power supply 1906 includes one or more buttons to operate the system, such as a power button 1918, a start button 1920, and a stop button 1922.

FIG. 13B shows a mask configured to wirelessly transfer to two patches with attached microneedles through inductive power transfer. The mask 1312 is placed over patches 1314 and 1316 attached to the skin of the subject, with the microneedles in the array inserted into the subcutaneous fat. The mask 1312 and the patches 1314 and 1316 are not connected by any wires or other tethers. The mask 1312 includes an on-board rechargeable battery 1318 electrically connected to a cable connector 1320. A removable plug 1322 can plug into the cable connector 1320 and a wall socket (not shown) to recharge the battery 1318. The cable connector 1320 is also electrically connected to an antenna 1324 in the mask 1324. The cable connector 1322 can include a switch or other electrical circuitry to charge the batter 1318 or provide an electrical current through the antenna 1324. The electrical current flowing through the antenna 1324 allows for wireless power transfer to the patches 1314 and 1316, which each include a second antenna.

Electrical power applied to the disposable patch via the overlying mask can be transformed into heat by means such as Joule (electrical resistive) heating or magnetically-induced eddy current heating. The applied power acts to raise the temperature of all of the microneedle tips to a temperature of 50-80° C., which is the temperature range known to melt subcutaneous fat. Alternatively, the applied power can raise the temperature of the microneedle tips to a temperature of about 33-60° C., which can also be used to liquefy or melt subcutaneous fat deposits. Applying this energy through the tips of microneedles allows for localized and focused application of the energy to the target area to reduce/cure/melt/dissolve targeted a fat layer.

The mask can include electronics, for example, a drive circuit, sealed within the mask material, to provide power, telemetry and sensing functionality, working in concert with the disposable patch or patches. The mask may have either an onboard power source, such as a rechargeable battery, or a cord that connects to an electrical power source or outlet.

The advent of mobile and wireless network has facilitated non-wired or even remote control of medical devices. For human subjects being treated with microneedle devices comprising a mask over the eyes, it can be difficult to adjust any operation by switches or handles. A computerized device with telemetry uplink can assist in asserting control of the device via touch or voice commands on a computer system. Such computerized devices with telemetry uplink can also allow users or physicians to remotely adjust device parameters for improved performance and treatment by the device. Therefore, in some embodiments according to any one of the devices described above, the device further comprises a telemetry uplink antenna configured to communicate with a computer system or a network. In some instances, it may be advantageous to control the device entirely on computer systems, such as to improve precision or for remote treatment of subjects with low physical mobility. In some embodiments according to any one of the devices described above, the device is operated using the computer system. As non-limitation examples, a computer system can comprise any one of server computers, personal computers, cellular phones, smartphones, computer tablets or personal digital assistants (PDA). In further non-limiting examples, a network can be the Internet, intranet, cellular network or cloud-based network.

Disclosed herein is a method to reduce fat deposits in close proximity to skin by using a microneedle treatment system. Some examples of these areas include, but not limited to, the fat pads below the eye (“baggy or puffy eye” appearance, blepharochalasis, dermatochalasis), along the jaw line (jowls), along the cheek smile lines (nasolabial folds), and below the chin (“double-chin” or sub-genial fat). The method is also able to address targeted deep layers of the skin.

In some embodiments, there is a method of tightening skin or reducing a subcutaneous fat deposit in a subject, comprising inserting a plurality of microneedles into a subject, wherein the tips of the microneedles are positioned within a subcutaneous fat deposit; and heating the tips of the microneedles using less than about 2.5 W of power, thereby melting fat within the subcutaneous fat deposit.

In another example of a method for tightening skin or reducing a facial fat deposit in a subject, the method includes inserting a plurality of microneedles into a subject, wherein the tips of the microneedles are positioned within a facial fat deposit; heating the tips of the microneedles, thereby melting fat within the subcutaneous fat deposit. In some embodiments, the facial fat deposit is a periorbital fat deposit (e.g., a postseptal fat deposit or a preseptal fat deposit), a jowl fat deposit, forehead fat deposit, lateral orbital fat deposit, malar fat deposit, or nasolabial fat deposit.

In a further example, there is a method of tightening skin or reducing a subcutaneous fat deposit of a subject, comprising: inserting the plurality of microneedles in any one of the systems described herein into the subject, wherein the tips of the microneedles are positioned within the subcutaneous fat deposit; and heating the tips of the microneedles, thereby melting fat within the facial fat area.

In accordance with any of the methods described herein, the tips of the microneedles can be heated by applying less than about 2.5 W of power to the microneedles to effect melting of the fat, such as about 0.05 W to about 0.5 W, about 0.5 W to about 1 W, about 1 W to about 1.5 W, about 1.5 W to about 2 W, or about 2 W to less than about 2.5 W of power. In certain examples, the tips of the microneedles can be heated by applying about 100 mW to about 500 mW of power to the microneedles, such as about 50 mW to about 100 mW, about 100 mW to about 200 mW, about 200 mW to about 300 mW, about 300 mW to about 400 mW, about 400 mW to about 500 mW of power, about 500 mW to about 600 mW, about 600 mW to about 700 mW, about 700 mW to about 800 mW, about 800 mW to about 900 mW, about 900 mW to about 1000 mW of power.

The safety enhancement of the methods in the present application can comprise application safer energy output profile over a longer treatment session. In some embodiments according to any one of the described methods, the tips of the microneedles are heated for about 1 minute to about 20 minutes. For example, in some embodiments, the tips of the microneedles are heated for about 1 minute to about 2 minutes, for about 2 minutes to about 3 minutes, for about 3 minutes to about 4 minutes, for about 4 minutes to about 5 minutes, for about 5 minutes to about 6 minutes, for about 6 minutes to about 7 minutes, for about 7 minutes to about 8 minutes, for about 8 minutes to about 9 minutes, for about 9 minutes to about 10 minutes, for about 10 minutes to about 11 minutes, for about 11 minutes to about 12 minutes, for about 12 minutes to about 13 minutes, for about 13 minutes to about 14 minutes, for about 14 minutes to about 15 minutes, for about 15 minutes to about 16 minutes, for about 16 minutes to about 17 minutes, for about 17 minutes to about 18 minutes, for about 18 minutes to about 19 minutes, or for about 19 minutes to about 20 minutes.

The microneedle treatment device described herein delivers a thermal output with enhanced safety for the subject. As opposed to using high heat to melting fats, the device described herein operates under a safer range of temperatures. The low power and low temperature required to melt subcutaneous fats with microneedle tips inserted directly into the subcutaneous fat deposits enhances device safety. In some embodiments the tips of the microneedles are heated to about 33° C. to about 65° C., such as about 30° C. to about 33° C., about 33° C. to about 35° C., about 35° C. to about 40° C., about 40° C. to about 45° C., about 45° C. to about 50° C., about 50° C. to about 55° C., about 55° C. to about 60° C., or about 60° C. to about 65° C.

The patch comprising the microneedle array is attached to the skin above the fat deposit by inserting the microneedles through the skin. The patch can include a skin-safe (and, preferably, thermally-resistant) adhesive on the side of the patch attached to the microneedles, allowing the patch to be secured to the skin. In some embodiments, a mask is placed over the patch. The mask may include the power supply, and energy can be transferred from the mask to the patch, wherein the transferred energy heats the tips of the microneedles. In some embodiments, the method comprises placing the mask over, around, or below an eye of a human subject, and over the patch. In some embodiments, the mask can cover the upper portion of the face (upper face mask), the lower portion of the face (lower face mask), the left side of the face, the right side of the face, or the entire face (full face mask). In some embodiments, the patch can be placed over one or more of lower eyelid, the nasolabial fold, or the jowl regions.

A built-in control that limits the maximum energy delivered and/or to regulate the temperature of the microneedle can reduce risks of overheating. To control the temperature of the microneedle tips, the patch or the mask can optionally include a temperature regulator and/or energy regulator configured to suspend heating of the microneedles if the temperature or energy exceeds a predetermined threshold. Accordingly, in some embodiments, the methods described herein include monitoring or controlling the temperature or heating of the tips of the microneedles, which may be performed, for example using a computer system.

Optionally, the system includes a telemetry uplink antenna configured to communicate with a computer system or a network. The computer system allows added convenience to the user, including operation of the system using a convenient interface. In some instances, it may be advantageous to control the device entirely on computer systems. As non-limitation examples, a computer system can comprise any one of server computers, personal computers, cellular phones, smartphones, computer tablets or personal digital assistants (PDA). In further non-limiting examples, a network can be the Internet, intranet, cellular network or cloud-based network.

The patch or patches can be applied by the user to the desired treatment region and pressed into place. The needle array pierces the skin, thereby holding the patch in place. The tips of the needles are in contact with the surface of or lodge into the underlying fat pockets.

After the patches are applied to the desired region, a thin, reusable overlying mask can be placed directly over the adhered patches. An example on the face would be a mask similar to those used to apply beauty skin products, for example, a full-face mask, an upper face/eye mask that covers only the region around the eyes, or a lower face mask. The mask can be made from a soft, flexible material, such as silicone, that feels pleasant on the skin and that allows cleaning with water. The mask can have cutouts to allow the user to see and to breathe comfortably.

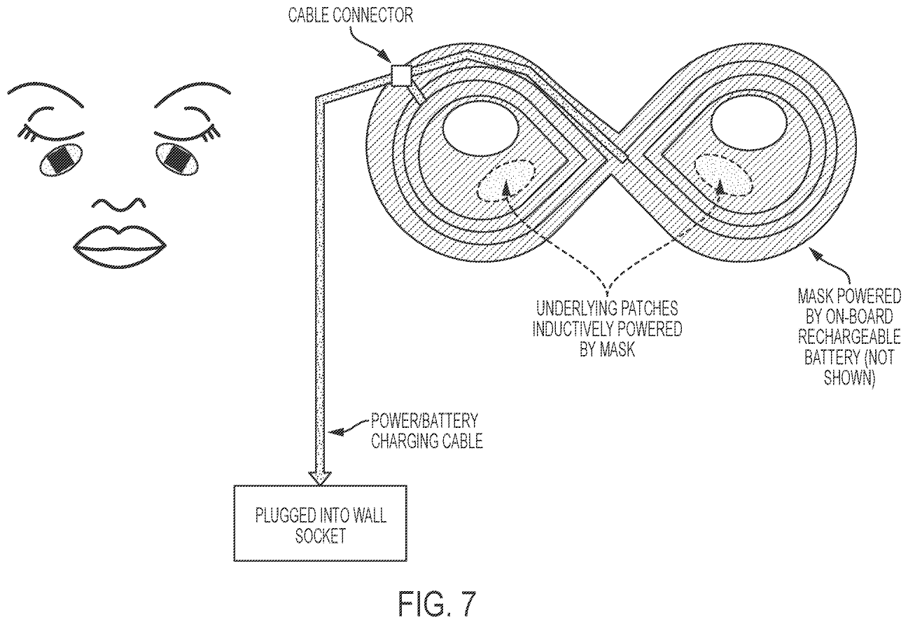

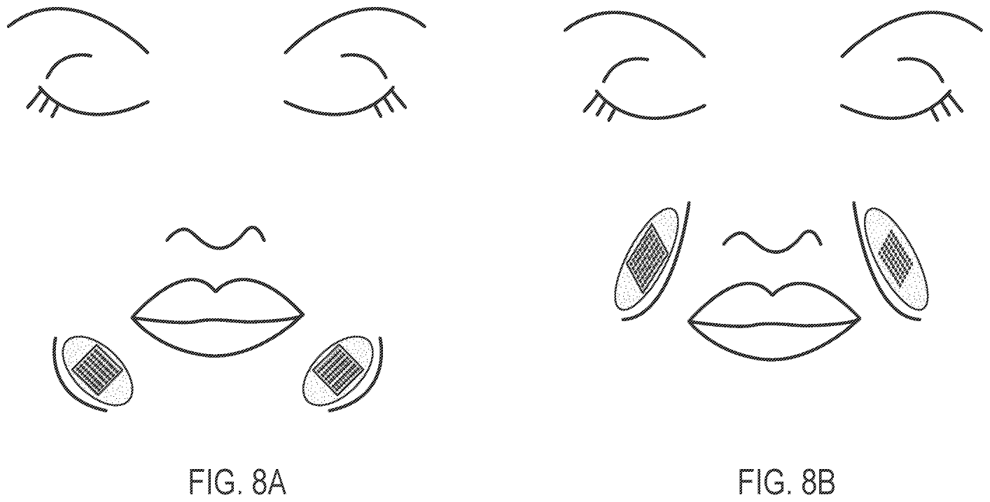

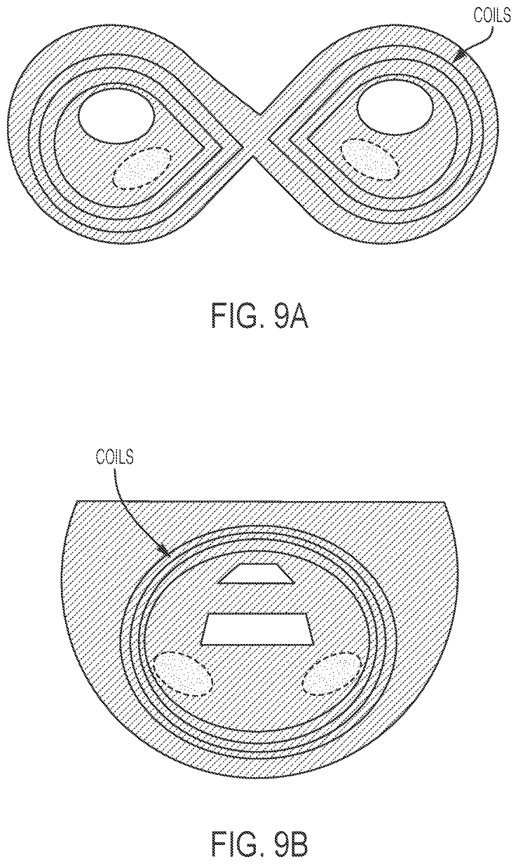

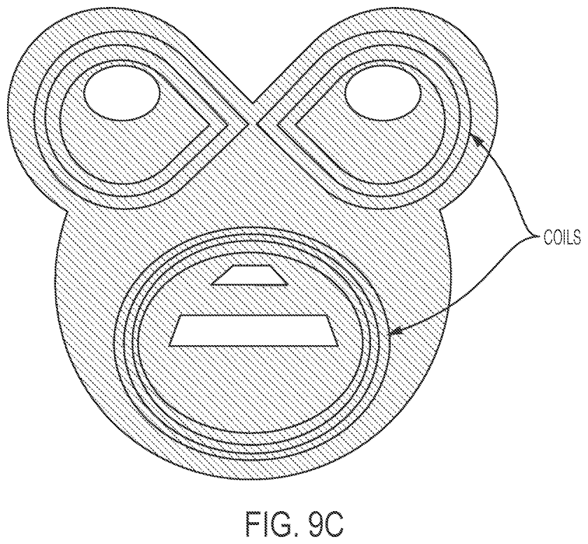

FIG. 7 schematically illustrates an overlying mask attached to a disposable patch to inductively power the disposable patch. Power to the overlying face masked can be plugged power or provided by an on-board battery (i.e., unplugged). In the illustrated example, the mast is powered directly by being plugged into a wall socket. The mask can also include a coil antenna for transferring power from the battery or external power outlet, to the disposable patches, by inductive power transfer (similar in operation to RFID or electric vehicle charging systems). The mask may have a second antenna, or use the power coil antenna, to provide communication functions via Bluetooth or Wi-Fi, in order to send data from the mask to the internet, a smartphone, or a computer.

The mask may also include a number of sensors, such as those to detect orientation, motion, and temperature, in order to detect the user's behavior and to monitor the heating functions of the disposable patch or patches. The user can use the control unit on the mask, or the smartphone, or the computer, to start, monitor and end the procedure. After treatment, the reusable mask can be removed, cleansed and preserved. The microneedle patches can be removed and disposed.

A software application, for example, residing on the user's smartphone or a computer, can be used to control the mask and disposable patches. The user may select different feature options, such as different temperature versus time profiles, according to the desired effect. The application may also track number of uses and provide suggestions to the user on which patch or patches to apply, and how frequently to do treatments. The application may also contain information on where to buy additional patches for future treatments, and enable live ordering of more disposable patches.

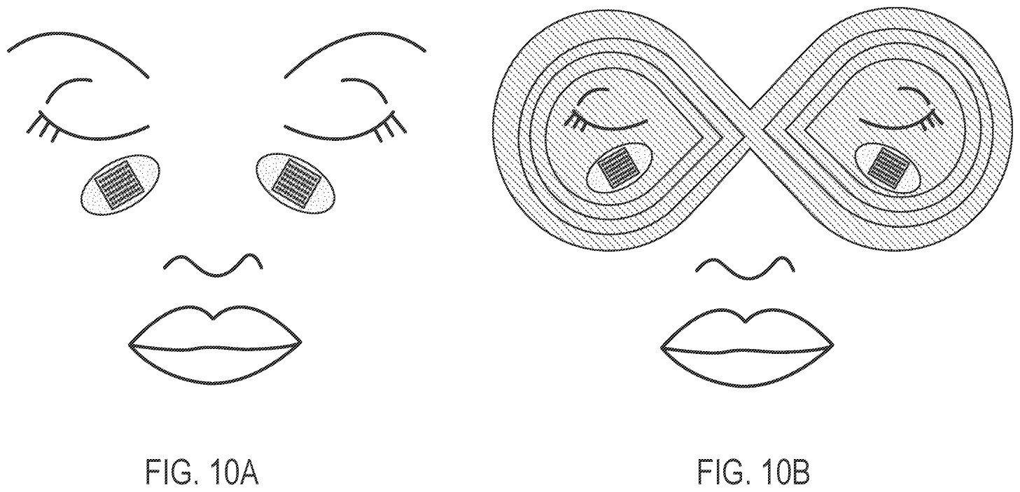

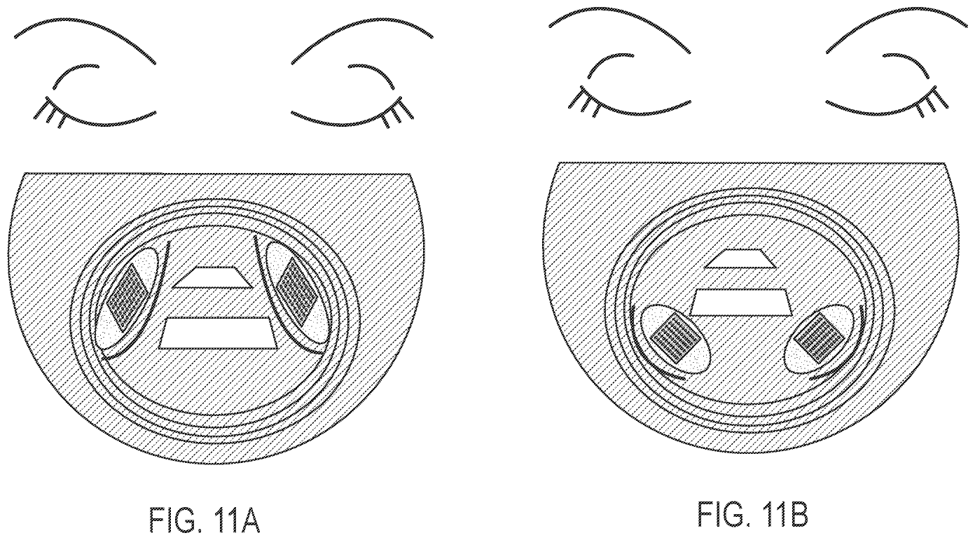

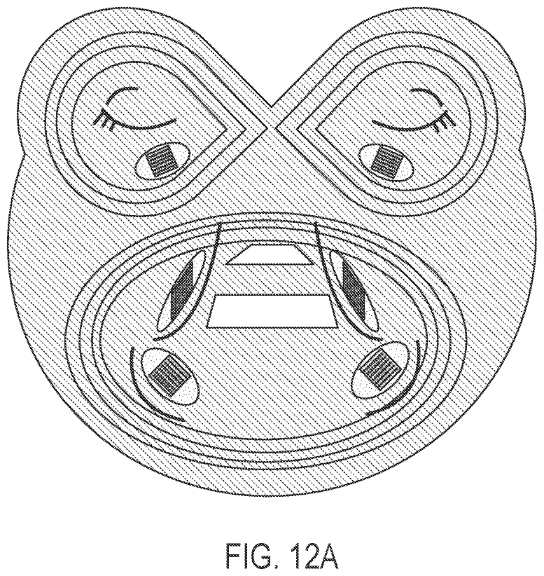

FIG. 10A schematically illustrates a method of applying one or more disposable patches in areas of fat deposits under eyes to reduce lower eyelid fat. FIG. 11A schematically illustrates a method of attaching a mask with one or more coils to multiple disposable patches in areas of fat deposits in nasolabial folds. FIG. 11B schematically illustrates a method of attaching a mask with one or more coils to multiple disposable patches in areas of fat deposits in the jowl region.

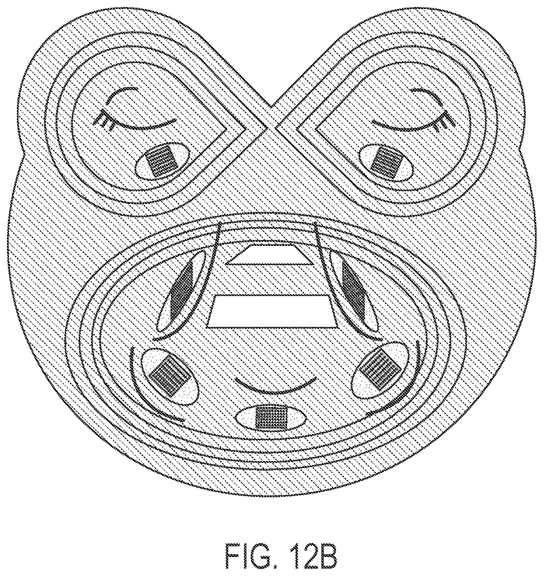

Further, the method can include a full-face mask with coils to multiple disposable patches in multiple areas of fat deposits. FIG. 12A schematically illustrates a method of attaching a full-face mask with coils to multiple disposable patches in multiple areas of fat deposits including under eye, nasolabial folds and jowls. FIG. 12B schematically illustrates a method of attaching a full-face mask with coils to multiple disposable patches in multiple areas of fat deposits including under the eye, nasolabial folds, jowls and under the chin area.

Melting of fat in subcutaneous fat deposits can be determined using methods known in the art, such as using the methods described in Zhang et al., Locally Induced Adipose Tissue Browning by Microneedle Patch for Obesity Treatment, ACS Nano, vol. 11, pp. 922-9230 (2017). For example, a fat sample can be stained using hematoxylin and eosin (H&E) and visualized for shrinkage and altered morphology of adipocytes.

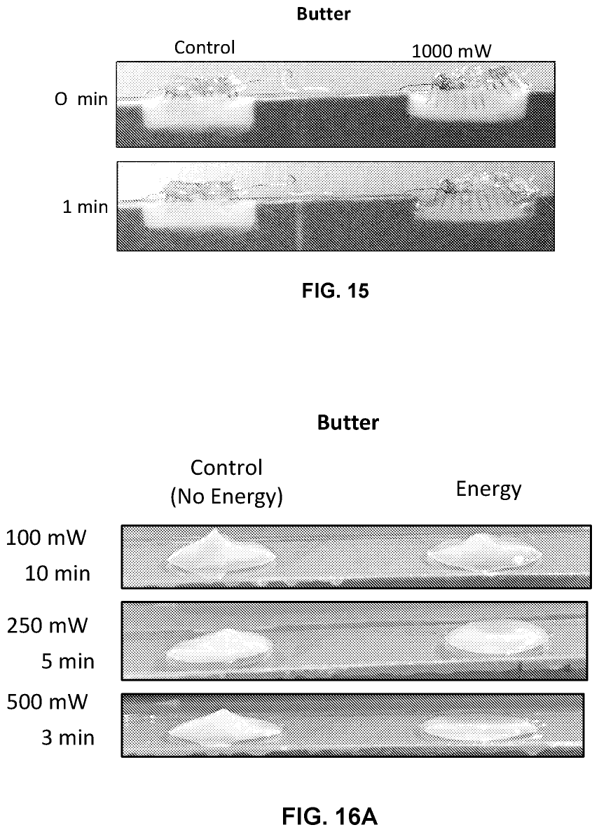

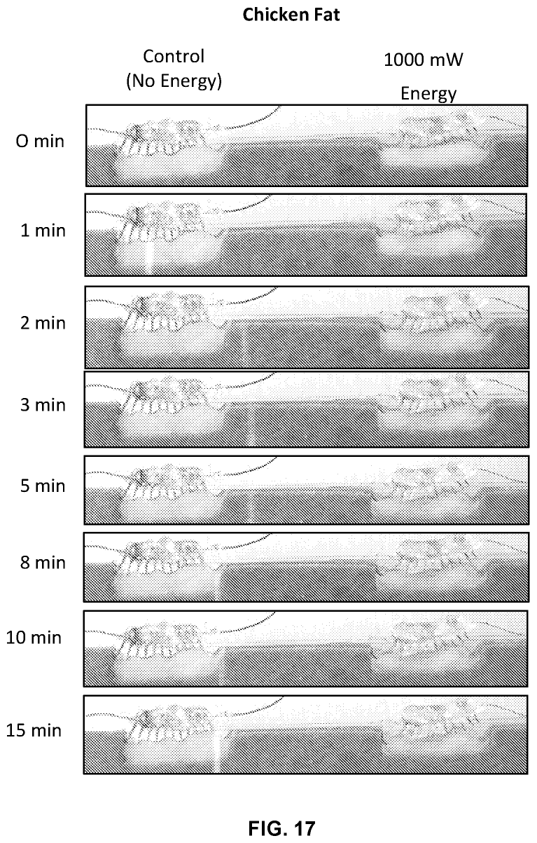

Melting of solid fat or any other suitable test substrate can also be monitored or assayed in vitro, for example to determine a desirable power level and/or time of heating microneedles, using a newly designed apparatus for use with a device or system comprising microneedles. The apparatus includes a first surface and a second surface, with a middle layer connecting the first surface and the second surface. The first surface and/or the second surface may comprise, for example, glass, metal, or other thermally resistant material. The middle layer can include any suitable material such as a polymeric foam or rubber. Preferably, the middle layer comprises a non-conductive material. The first and second surfaces are parallel to each other, and the middle layer includes a well that is visible through a transparent region on the first surface or the second surface. A test substrate, such as a solid fat, is deposited in the well, and is therefore visible through the transparent region. The well is also configured to receive the plurality of microneedles from the device. The microneedles are configured to be heated using a power source, and the melting of the fat can be monitored with the tips of the microneedles inserted into the test substrate, such as the solid fat. Melting of the test substrate (e.g., solid fat) may be monitored at one or more time points and/or one or more different power levels. An exemplary device is shown in FIG. 15 and FIG. 17.

Suitable test substrates that can be used with the apparatus for monitoring melting of the test substrate include solid fat (which may comprise saturated fat), a polymer, a rubber, or any other solid or semi-solid substrate.

The apparatus for monitoring melting can be used to qualitatively or quantitatively determine the degree of melting. For example, the transparent region in the surface of the apparatus allows for visual observation of melting of the test substrate (e.g., the solid fat). In certain embodiments, the transparent region includes one or more graduated markings, and the portion of melted substrate and un-melted substrate can be quantitatively measured.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

### EXEMPLARY EMBODIMENTS

The following embodiments are exemplary of the present invention and should not be considered limiting.

### Embodiment 1

A microneedle treatment system, comprising:

a microneedle array attached to a patch, the microneedle array comprising a plurality of fixed-length microneedles, the microneedles comprising an insulated shaft and an uninsulated tip; and

a power supply configured to heat the plurality of microneedles using less than about 2.5 W of power.

### Embodiment 2

The system embodiment 1, wherein the power supply is configured to heat the plurality of microneedles using about 100 mW to about 500 mW of power.

### Embodiment 3

A microneedle treatment system, comprising:

a microneedle array attached to a patch, the microneedle array comprising a plurality of fixed-length microneedles, the microneedles comprising an insulated shaft and an uninsulated tip; and

a power supply configured to heat the plurality of microneedles using about 50 mW of power or less per microneedle.

### Embodiment 4

The system of embodiment 3, wherein the power supply is configured to heat the plurality of microneedles using about 1 mW to about 50 mW of power per microneedle.

### Embodiment 5

A microneedle treatment system, comprising:

a patch comprising a dome-shape body comprising a top and a base, and a microneedle array comprising a plurality of microneedles housed within a cavity within the dome-shaped body and attached to an inner surface of the dome-shaped body, wherein the form of the body can be changed into a substantially flat configuration that results in at least a portion of the microneedles to be repositioned from within the cavity to below the base; and

a power supply configured to heat the plurality of microneedles.

### Embodiment 6

The system of embodiment 5, wherein the microneedles are fixed-length microneedles.

### Embodiment 7

The system of embodiment 5 or 6, wherein the microneedles comprising an insulated shaft and an uninsulated tip.

### Embodiment 8

The system of any one of embodiments 5-7, wherein the power supply is configured to heat the plurality of microneedles using less than about 2.5 W of power.

### Embodiment 9

The system of embodiment 8, wherein the power supply is configured to heat the plurality of microneedles using about 100 mW to about 1000 mW of power.

### Embodiment 10

The system of any one of embodiments 5-9, wherein the power supply is configured to heat the plurality of microneedles using about 50 mW of power or less per microneedle.

### Embodiment 11

The system of embodiment 10, wherein the power supply is configured to heat the plurality of microneedles using about 1 mW to about 50 mW of power per microneedle.

### Embodiment 12

The system of any one of embodiments 5-11, wherein the base comprises a lip.

### Embodiment 13

The system of any one of embodiments 5-12, wherein the base comprises an adhesive.

### Embodiment 14

The system of any one of embodiments 1-13, wherein the microneedles are about 2 mm to about 8 mm in length.

### Embodiment 15

The system of any one of embodiments 1-14, wherein the microneedles are about 3 to about 4 mm in length.

### Embodiment 16

The system of any one of embodiments 1-15, wherein the uninsulated tip is about 0.5 mm to about 1.0 mm in length.

### Embodiment 17

The system of any one of embodiments 1-16, wherein the shaft of the microneedles is about 50 μm to about 500 μm in diameter.

### Embodiment 18

The system of any one of embodiments 1-17, wherein the plurality of microneedles comprises about 3 microneedles to about 100 microneedles.

### Embodiment 19

The system of any one of embodiments 1-18, wherein the power supply is configured to heat the tips of the microneedles from about 33° C. to about 60° C.

### Embodiment 20

The system of any one of embodiments 1-19, wherein the plurality of microneedles is heated using a direct current energy.

### Embodiment 21

The system of any one of embodiments 1-19, wherein the plurality of microneedles is heated using a radiofrequency energy.

### Embodiment 22

The system of any one of embodiments 1-21, wherein the system is a hands-free system.

### Embodiment 23

The system of any one of embodiments 1-4 and 14-22, wherein the patch comprises an adhesive.

### Embodiment 24

The system of any one of embodiments 1-4 and 14-23, wherein the patch is crescent-shaped, semi-circular, triangular, square, or rectangular.

### Embodiment 25

The system of any one of embodiments 1-24, wherein the power supply comprises a battery.

### Embodiment 26

The system of any one of embodiments 1-25, wherein the power supply is connected to the microneedle array through a wire.

### Embodiment 27

The system of any one of embodiments 1-26, wherein the power supply is wirelessly connected to the microneedle array.

### Embodiment 28

The system of embodiment 27, wherein the patch comprises a first antenna electrically connected to the microneedle array, wherein the power supply comprises a second antenna, and wherein the power supply powers the microneedle array through inductive power transfer.

### Embodiment 29

The system of any one of embodiments 1-28, comprising a mask comprising the power supply, wherein the mask is configured to be placed over the patch.

### Embodiment 30

The system of embodiment 29, wherein the mask is configured to be placed over, around, or below an eye of a human subject, and over the patch.

### Embodiment 31

The system of any one of embodiments 1-30, wherein the patch or the mask comprises a temperature configured to suspend heating of the microneedles if the temperature goes above a predetermined threshold.

### Embodiment 32

The system of any one of embodiments 1-31, further comprising a telemetry uplink antenna configured to communicate with a computer system or a network.

### Embodiment 33

The system of embodiment 32, wherein the system is operated using the computer system.

### Embodiment 34

A method of reducing a subcutaneous fat deposit in a subject, comprising:

inserting a plurality of microneedles into the subject, wherein the tips of the microneedles are positioned within the subcutaneous fat deposit or on the surface of the subcutaneous fat deposit; and

heating the tips of the microneedles using less than about 2.5 W of power, thereby melting fat within the subcutaneous fat deposit.

### Embodiment 35

The method of embodiment 34, wherein heating the tips of the microneedles comprises applying about 100 mW to about 500 mW of power to the microneedles.

### Embodiment 36

A method of reducing a subcutaneous fat deposit in a subject, comprising:

inserting a plurality of microneedles into the subject, wherein the tips of the microneedles are positioned within the subcutaneous fat deposit or on the surface of the subcutaneous fat deposit; and

heating the tips of the microneedles using about 50 mW of power or less per microneedle, thereby melting fat within the subcutaneous fat deposit.

### Embodiment 37

The method of embodiment 36, wherein heating the tips of the microneedles comprises applying about 1 mW to about 50 mW of power per microneedle.

### Embodiment 38

A method of reducing a facial fat deposit in a subject, comprising:

inserting a plurality of microneedles into the subject, wherein the tips of the microneedles are positioned within the facial fat deposit or on the surface of the facial fat deposit; and

heating the tips of the microneedles, thereby melting fat within the facial fat deposit.

### Embodiment 39

The method of embodiment 38, wherein the facial fat deposit is a periorbital postseptal fat deposit, a periorbital preseptal fat deposit, or a jowl fat deposit.

### Embodiment 40

A method of reducing a subcutaneous fat deposit in a subject, comprising:

positioning a dome-shaped patch comprising a plurality of microneedles on a target skin area above the subcutaneous fat deposit;

reconfiguring the dome-shaped patch into a substantially flat configuration, thereby inserting the tips of the microneedles into the into the subcutaneous fat deposit; and

heating the tips of the microneedles, thereby melting fat within the subcutaneous fat deposit.

### Embodiment 41

The method of embodiment 40, wherein reconfiguring the dome-shaped patch comprises applying pressure to the top of the dome-shaped patch.

### Embodiment 42

The method of embodiment 40 or 41, wherein the target skin area is stretched upon reconfiguring the dome-shaped patch into the substantially flat configuration.

### Embodiment 43

The method of any one of embodiments 38-42, wherein heating the tips of the microneedles comprises applying less than about 2.5 W of power to the microneedles.

### Embodiment 44

The method of embodiment 43, wherein heating the tips of the microneedles comprises applying about 100 mW to about 500 mW of power to the microneedles.

### Embodiment 45

The method of any one of embodiments 38-44, wherein heating the tips of the microneedles comprises applying about 50 mW of power or less per microneedle.

### Embodiment 46

The method of any one of embodiments 38-45, wherein heating the tips of the microneedles comprises applying about 1 mW to about 50 mW of power per microneedle.

### Embodiment 47

The method of any one of embodiments 34-46, wherein the tips of the microneedles are heated for about 1 minute to about 20 minutes.

### Embodiment 48

The method of any one of embodiments 34-47, wherein heating the tips of the microneedles comprises applying a direct current energy to the microneedles.

### Embodiment 49

The method of any one of embodiments 34-47, wherein heating the tips of the microneedles comprises applying a radiofrequency energy to the microneedles.

### Embodiment 50

The system of any one of embodiments 34-49, wherein the plurality of microneedles comprises about 3 microneedles to about 100 microneedles.

### Embodiment 51

The method of any one of embodiments 34-50, wherein the tips of the microneedles are heated to about 33° C. to about 60° C.

### Embodiment 52

The method of any one of embodiments 34-51, wherein the microneedles comprise an insulated shaft, and wherein the tips of the microneedles are uninsulated.

### Embodiment 53

The method of any one of embodiments 34-52, comprising attaching a patch comprising the plurality of microneedles to skin above the fat deposit.

### Embodiment 54

The method of embodiment 53, comprising placing a mask over the patch.

### Embodiment 55

The method of embodiment 54, comprising wirelessly transferring energy from the mask to the patch, wherein the transferred energy heats the tips of the microneedles.

### Embodiment 56

The method of any one of embodiments 34-55, comprising controlling the heating of the tips of the microneedles using a computer system.

### Embodiment 57

A method of reducing a subcutaneous fat deposit in a subject, comprising:

inserting the plurality of microneedles of the system of any one of embodiments 1-33 into the subject, wherein the tips of the microneedles are positioned within the subcutaneous fat deposit or on a surface of the subcutaneous fat deposit; and

heating the tips of the microneedles, thereby melting fat within the subcutaneous fat deposit.

### Embodiment 58

The method of embodiment 57, wherein the subcutaneous fat deposit is a subcutaneous facial fat deposit.

### Embodiment 59

The method of embodiment 57 or 58, wherein the subcutaneous fat deposit is a periorbital postseptal fat deposit or a periorbital preseptal fat deposit.

### Embodiment 60

An apparatus for monitoring melting of a test substrate, comprising:

a first surface and a second surface, the first surface comprising a transparent region, wherein the first surface and the second surface are parallel;

a middle layer connecting the first surface to the second surface, the middle layer comprising a well containing the test substrate, wherein the test substrate is visible through the transparent region of the first surface, and wherein the well is configured to receive tips of the plurality of microneedles.

### Embodiment 61

The apparatus of embodiment 60, wherein the first surface or the second surface comprises glass or thermally-resistant material.

### Embodiment 62

The apparatus of embodiment 60 or 61, wherein the middle layer comprises a polymeric foam or rubber.

### Embodiment 63

The apparatus of any one of embodiments 60-62, further comprising a device comprising a plurality of microneedles that are inserted in the test substrate or positioned on the surface of the test substrate.

### Embodiment 64

The apparatus of embodiment 63, wherein the microneedles are configured to be heated using a power source.

### Embodiment 65

The apparatus of any one of embodiments 60-64, wherein the transparent region comprises one or more graduated markers for quantitative analysis.

### Embodiment 66

The apparatus of any one of embodiments 60-65, wherein the test substrate is a solid fat.

### Embodiment 67

A method of monitoring melting of a test substrate, comprising:

applying energy to a plurality of microneedles inserted into the test substrate using the apparatus of any one of embodiments 60-66; and

monitoring melting of the test substrate.

### Embodiment 68

The method of embodiment 67, wherein monitoring melting of the test substrate comprises qualitatively determining the degree of melting of the test substrate.

### Embodiment 69

The method of embodiment 67, wherein monitoring melting of the test substrate comprises quantitatively determining the degree of melting of the test substrate.

### Embodiment 70

The method of any one of embodiments 67-69, comprising monitoring the melting of the solid fat at a plurality of different power levels.

### Embodiment 70

The method of any one of embodiments 67-70, comprising monitoring the melting of the solid fat at a plurality of different time points.

### Embodiment 71

A microneedle treatment system comprising:

a disposable patch comprising a microneedle array comprising a plurality of microneedles; and

an overlying mask configured to be placed directly over the disposable patch, the mask comprising:

drive circuitry configured to deliver energy into the microneedle array;

a sensor configured for localized sensing; and

a telemetry uplink to a smartphone, a computer or a computer network.

### Embodiment 72

The system of embodiment 71, wherein each of the plurality of microneedles has a diameter smaller than 200 μm.

### Embodiment 73

The system of embodiment 71, wherein each of the plurality of microneedles has a length between 100 μm and 3500 μm.

### Embodiment 74

The system of embodiment 71, wherein the disposable patch further comprises a micro-coil.

### Embodiment 75

The system of embodiment 71, wherein the mask comprises a soft, flexible material.

### Embodiment 76

The system of embodiment 71, wherein the mask is reusable.

### Embodiment 77

The system of embodiment 71, wherein the mask further comprises a coil antenna to transfer power to the disposable patch by inductive power transfer.

### Embodiment 78

The system of embodiment 77, wherein the mask further comprises a second antenna to send data from the mask to a smartphone, a computer or a computer network.

### Embodiment 79

The system of embodiment 71, wherein each of the plurality of microneedles comprises a material that is thermally conductive and biologically compatible for subcutaneous use.

### Embodiment 80

The system of embodiment 71, wherein each of the plurality of microneedles comprises a coating that is non-conductive and biologically compatible for subcutaneous use.

### Embodiment 81

The system of embodiment 71, wherein the microneedle array comprises multiple sub-arrays disposed onto multiple rigid substrates to form a semi-flexible substrate.

### Embodiment 82

A method to tighten skin and or reduce fat deposits directly under or in close proximity to skin by using a microneedle treatment system, the method comprising:

applying a disposable patch comprising a microneedle array comprising a plurality of microneedles to a targeted treatment area;

placing an overlying mask directly over the disposable patch;

delivering energy into the microneedle array;

monitoring a heating function of the disposable patch; and

transmitting data from the mask to a smartphone, a computer or a computer network by using a telemetry uplink.

### Embodiment 83

The method of embodiment 82, further comprising receiving inductive power with a micro-coil on the disposable patch.

### Embodiment 84

The method of embodiment 82, further comprising applying the energy through tips of the microneedles to a targeted area to tighten skin layer and or reduce a targeted fat layer.

### Embodiment 85

The method of embodiment 82, further comprising delivering power inductively to the disposable patch from a coil antenna in the mask.

### Embodiment 86

The method of embodiment 82, further comprising controlling the mask and the disposable patch by a software application.

### Embodiment 87

The method of embodiment 86, further comprising sending data from the mask to a smartphone, a computer or a computer network by a second antenna in the mask.

### Embodiment 88

The method of embodiment 82, further comprising conforming the disposable patch to a large area to be treated by using a microneedle array comprising multiple sub-arrays disposed onto multiple rigid substrates to form a semi-flexible substrate.

### Embodiment 89

The method of embodiment 82, wherein the step of applying the disposable patch comprising applying the disposable patch under a pair of eyes of a user in areas of fat deposits.

### Embodiment 90

The method of embodiment 82, wherein the step of applying the disposable patch comprising applying the disposable patch to a jowl region of a user in areas of fat deposits.

### Embodiment 91

The method of embodiment 82, wherein the step of applying the disposable patch comprising applying the disposable patch to a nasolabial fold region of a user in areas of fat deposits.

### EXAMPLE

### Determination of Power and Time Required to Liquefy Fat

This experiment describes the approximate measurement of energy and time needed to melt certain fats that approximate human facial subcutaneous fats. Fats with higher melting points will require application of a higher energy output and/or a longer time to adequately melt. Generally, the melting points of fats depending on the fatty acid composition, and are correlated with the degree of saturation of the fatty acids.

The melting points of human fats vary based on their location in the body (See Schmidt-Nielsen, Melting Points of Human Fats as Related to their Location in the Body, Acta Physiologica, vol. 12, pp. 123-129 (1946)). Additionally, the closer the fat is to the surface of the skin, the lower the relative melting point, and human skin mean temperature is in the range of 32-35° C. Human fats are similar in composition as chicken fats, with a similar carotene content and saturated fat content (29% saturated fat content in both human and chicken fat), suggesting similar melting temperatures for the fats. Chicken fat, with a melting point of 33-40° C. (See Houghton, The Effect of Low Temperatures on Ground Chicken Meat, Ind. Eng. Chem., vol. 3, no. 7, pp. 497-506 (1911)), provides a satisfactory model for determining the energy required for liquefying human subcutaneous fats. Butter has a melting point of about 32-35° C. (Schäffer et al., Melting Properties of Butter Fat and the Consistency of Butter, Journal of Thermal Analysis and calorimetry, vo. 64, no. 2, pp. 659-669 (2001)), overlaps into the range of human skin temperature and melting points of chicken fat, is easily accessible, and is similarly a useful model to show effect of liquefaction. To determine the amount of energy output and time needed to liquefy various kinds of fats, microneedles connected by an electrically conducting wire was inserted into a mass of solid chicken fat or butter. Electrical power was passed through the conducting wire for various periods of time, and fat melting was monitored.

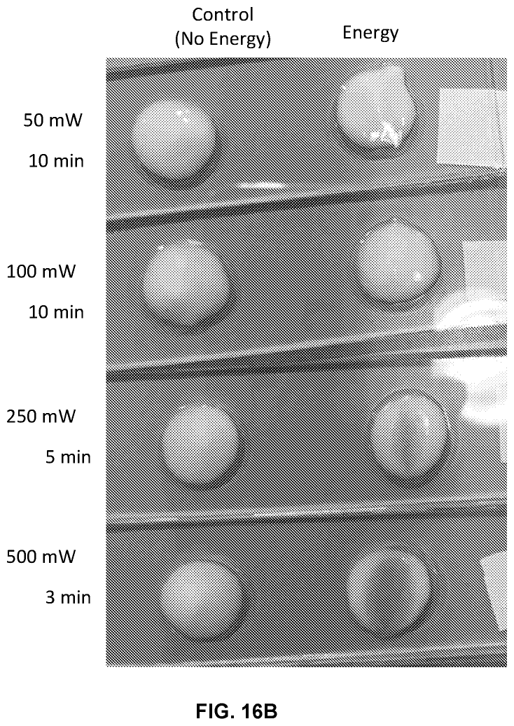

Butter was subjected to about 1000 mW of energy (2.1 volts at 0.46 amps) for 1 minute, compared to a control that did not have energy applied. As shown in FIG. 15, the butter began to melt at the 1 minute interval (about 40-50% liquefaction). At the lower energy range, by subjecting the butter to about 50 mW (0.5 V at 0.1 amps), the butter only slightly melted after 10 minutes. Using different powers for different lengths of time, butter was found to melt after applying 100 mW for 10 minutes, 250 mW for 5 minutes, and 500 mW for 3 minutes (see FIGS. 16A and 16B).

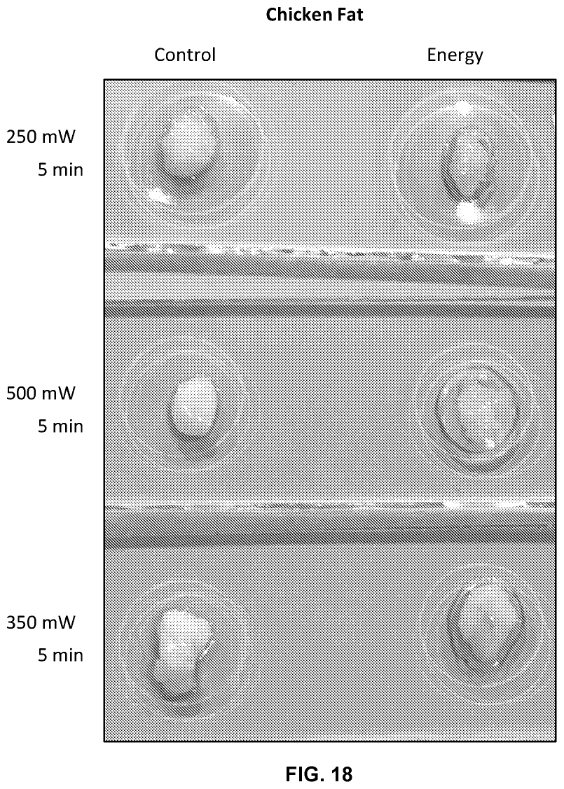

Chicken fat was subjected to about 1000 mW (2.1 V at 0.47 amps) or 50 mW (0.5 V at 0.1 amps) of energy for a time course of 15 minutes. At 1000 mW, the chick fat began to melt after about 2-3 minutes (FIG. 17), with about 30-40% liquefaction after about 5 minutes. No significant melting was noticed after applying 50 mW of energy for 15 minutes (data not shown). In a separate experiment, 250 mW, 350 mW, or 500 mW of energy was applied to chicken fat for 5 minutes, which resulted in melting of the chicken fat (see FIG. 18).

This example demonstrates that it is practicable to cause melting or liquefaction of fats with similar characteristics as chicken fats, using energy as low as 250 mW to 500 mW, when applied for five minutes or longer. Because of its similarity of composition and saturation profiles as chicken fats, human subcutaneous fats are expected to display similar melting characteristics.

What is claimed is:

1. A microneedle treatment system, comprising:
a microneedle array attached to a patch, the microneedle array comprising a plurality of fixed-length microneedles, the microneedles comprising an insulated shaft and an uninsulated tip; and
a power supply configured to heat the plurality of microneedles using less than about 2.5 W of power.

2. The system claim 1, wherein the power supply is configured to heat the plurality of microneedles using about 100 mW to about 1000 mW of power.

3. A microneedle treatment system, comprising:
a microneedle array attached to a patch, the microneedle array comprising a plurality of fixed-length microneedles, the microneedles comprising an insulated shaft and an uninsulated tip; and
a power supply configured to heat the plurality of microneedles using about 50 mW of power or less per microneedle.

4. The system of claim 3, wherein the power supply is configured to heat the plurality of microneedles using about 1 mW to about 50 mW of power per microneedle.

5. A microneedle treatment system, comprising:
a patch comprising a dome-shape body comprising a top and a base, and a microneedle array comprising a plurality of microneedles housed within a cavity within the dome-shaped body and attached to an inner surface of the dome-shaped body, wherein the form of the body can be changed into a substantially flat configuration that results in at least a portion of the microneedles to be repositioned from within the cavity to below the base; and
a power supply configured to heat the plurality of microneedles.

6. The system of claim 5, wherein the microneedles are fixed-length microneedles.

7. The system of claim 5 or 6, wherein the microneedles comprising an insulated shaft and an uninsulated tip.

8. The system of any one of claims 5-7, wherein the power supply is configured to heat the plurality of microneedles using less than about 2.5 W of power.

9. The system of claim 8, wherein the power supply is configured to heat the plurality of microneedles using about 100 mW to about 1000 mW of power.

10. The system of any one of claims 5-9, wherein the power supply is configured to heat the plurality of microneedles using about 50 mW of power or less per microneedle.

11. The system of claim 10, wherein the power supply is configured to heat the plurality of microneedles using about 1 mW to about 50 mW of power per microneedle.

12. The system of any one of claims 5-11, wherein the base comprises a lip.

13. The system of any one of claims 5-12, wherein the base or the inner surface comprises an adhesive.

14. The system of any one of claims 1-13, wherein the microneedles are about 2 mm to about 8 mm in length.

15. The system of any one of claims 1-14, wherein the microneedles are about 3 to about 4 mm in length.

16. The system of any one of claims 1-15, wherein the uninsulated tip is about 0.5 mm to about 1.0 mm in length.

17. The system of any one of claims 1-16, wherein the shaft of the microneedles is about 50 μm to about 500 μm in diameter.

18. The system of any one of claims 1-17, wherein the plurality of microneedles comprises about 3 microneedles to about 100 microneedles.

19. The system of any one of claims 1-18, wherein the power supply is configured to heat the tips of the microneedles from about 33° C. to about 60° C.

20. The system of any one of claims 1-19, wherein the plurality of microneedles is heated using a direct current energy.

21. The system of any one of claims 1-19, wherein the plurality of microneedles is heated using a radiofrequency energy.

22. The system of any one of claims 1-21, wherein the system is a hands-free system.

23. The system of any one of claims 1-4 and 14-22, wherein the patch comprises an adhesive.

24. The system of any one of claims 1-4 and 14-23, wherein the patch is crescent-shaped, semi-circular, triangular, square, or rectangular.

25. The system of any one of claims 1-24, wherein the power supply comprises a battery.

26. The system of any one of claims 1-25, wherein the power supply is connected to the microneedle array through a wire.

27. The system of any one of claims 1-26, wherein the power supply is wirelessly connected to the microneedle array.

28. The system of claim 27, wherein the patch comprises a first antenna electrically connected to the microneedle array, wherein the power supply comprises a second antenna, and wherein the power supply powers the microneedle array through inductive power transfer.

29. The system of any one of claims 1-28, comprising a mask comprising the power supply, wherein the mask is configured to be placed over the patch.

30. The system of claim 29, wherein the mask is configured to be placed over, around, or below an eye of a human subject, and over the patch.

31. The system of any one of claims 1-30, wherein the patch or the mask comprises a temperature configured to suspend heating of the microneedles if the temperature goes above a predetermined threshold.

32. The system of any one of claims 1-31, further comprising a telemetry uplink antenna configured to communicate with a computer system or a network.

33. The system of claim 32, wherein the system is operated using the computer system.

34. A method of reducing a subcutaneous fat deposit in a subject, comprising:
inserting a plurality of microneedles into the subject, wherein the tips of the microneedles are positioned within the subcutaneous fat deposit or on the surface of the subcutaneous fat deposit; and
heating the tips of the microneedles using less than about 2.5 W of power, thereby melting fat within the subcutaneous fat deposit.

35. The method of claim 34, wherein heating the tips of the microneedles comprises applying about 100 mW to about 1000 mW of power to the microneedles.

36. A method of reducing a subcutaneous fat deposit in a subject, comprising:
inserting a plurality of microneedles into the subject, wherein the tips of the microneedles are positioned within the subcutaneous fat deposit or on the surface of the subcutaneous fat deposit; and
heating the tips of the microneedles using about 50 mW of power or less per microneedle, thereby melting fat within the subcutaneous fat deposit.

37. The method of claim 36, wherein heating the tips of the microneedles comprises applying about 1 mW to about 50 mW of power per microneedle.

38. A method of reducing a facial fat deposit in a subject, comprising:
inserting a plurality of microneedles into the subject, wherein the tips of the microneedles are positioned within the facial fat deposit or on the surface of the facial fat deposit; and
heating the tips of the microneedles, thereby melting fat within the facial fat deposit.

39. The method of claim 38, wherein the facial fat deposit is a periorbital postseptal fat deposit, a periorbital preseptal fat deposit, or a jowl fat deposit.

40. A method of reducing a subcutaneous fat deposit in a subject, comprising:
positioning a dome-shaped patch comprising a plurality of microneedles on a target skin area above the subcutaneous fat deposit;
reconfiguring the dome-shaped patch into a substantially flat configuration, thereby inserting the tips of the microneedles into the into the subcutaneous fat deposit; and
heating the tips of the microneedles, thereby melting fat within the subcutaneous fat deposit.

41. The method of claim 40, wherein reconfiguring the dome-shaped patch comprises applying pressure to the top of the dome-shaped patch.

42. The method of claim 40 or 41, wherein the target skin area is stretched upon reconfiguring the dome-shaped patch into the substantially flat configuration.

43. The method of any one of claims 38-42, wherein heating the tips of the microneedles comprises applying less than about 2.5 W of power to the microneedles.

44. The method of claim 43, wherein heating the tips of the microneedles comprises applying about 100 mW to about 500 mW of power to the microneedles.

45. The method of any one of claims 38-44, wherein heating the tips of the microneedles comprises applying about 50 mW of power or less per microneedle.

46. The method of any one of claims 38-45, wherein heating the tips of the microneedles comprises applying about 1 mW to about 50 mW of power per microneedle.

47. The method of any one of claims 34-46, wherein the tips of the microneedles are heated for about 1 minute to about 20 minutes.

48. The method of any one of claims 34-47, wherein heating the tips of the microneedles comprises applying a direct current energy to the microneedles.

49. The method of any one of claims 34-47, wherein heating the tips of the microneedles comprises applying a radiofrequency energy to the microneedles.

50. The system of any one of claims 34-49, wherein the plurality of microneedles comprises about 3 microneedles to about 100 microneedles.

51. The method of any one of claims 34-50, wherein the tips of the microneedles are heated to about 33° C. to about 60° C.

52. The method of any one of claims 34-51, wherein the microneedles comprise an insulated shaft, and wherein the tips of the microneedles are uninsulated.

53. The method of any one of claims 34-52, comprising attaching a patch comprising the plurality of microneedles to skin above the fat deposit.

54. The method of claim 53, comprising placing a mask over the patch.

55. The method of claim 54, comprising wirelessly transferring energy from the mask to the patch, wherein the transferred energy heats the tips of the microneedles.

56. The method of any one of claims 34-55, comprising controlling the heating of the tips of the microneedles using a computer system.

57. A method of reducing a subcutaneous fat deposit in a subject, comprising:
inserting the plurality of microneedles of the system of any one of claims 1-33 into the subject, wherein the tips of the microneedles are positioned within the subcutaneous fat deposit or on a surface of the subcutaneous fat deposit; and
heating the tips of the microneedles, thereby melting fat within the subcutaneous fat deposit.

58. The method of claim 57, wherein the subcutaneous fat deposit is a subcutaneous facial fat deposit.

59. The method of claim 57 or 58, wherein the subcutaneous fat deposit is a periorbital postseptal fat deposit or a periorbital preseptal fat deposit.

60. An apparatus for monitoring melting of a test substrate, comprising:
a first surface and a second surface, the first surface comprising a transparent region, wherein the first surface and the second surface are parallel;
a middle layer connecting the first surface to the second surface, the middle layer comprising a well containing the test substrate, wherein the test substrate is visible through the transparent region of the first surface, and wherein the well is configured to receive tips of the plurality of microneedles.

61. The apparatus of claim 60, wherein the first surface or the second surface comprises glass or thermally-resistant material.

62. The apparatus of claim 60 or 61, wherein the middle layer comprises a polymeric foam or rubber.

63. The apparatus of any one of claims 60-62, further comprising a device comprising a plurality of microneedles that are inserted in the test substrate or positioned on the surface of the test substrate.

64. The apparatus of claim 63, wherein the microneedles are configured to be heated using a power source.

65. The apparatus of any one of claims 60-64, wherein the transparent region comprises one or more graduated markers for quantitative analysis.

66. The apparatus of any one of claims 60-65, wherein the test substrate is a solid fat.

67. A method of monitoring melting of a test substrate, comprising:
applying energy to a plurality of microneedles inserted into the test substrate using the apparatus of any one of claims 60-66; and
monitoring melting of the test substrate.

68. The method of claim 67, wherein monitoring melting of the test substrate comprises qualitatively determining the degree of melting of the test substrate.

69. The method of claim 67, wherein monitoring melting of the test substrate comprises quantitatively determining the degree of melting of the test substrate.

70. The method of any one of claims 67-69, comprising monitoring the melting of the solid fat at a plurality of different power levels.

71. The method of any one of claims 67-70, comprising monitoring the melting of the solid fat at a plurality of different time points.
